(19) United States
(12) Patent Application Publication
(10) Pub. No.: US 2023/0240584 A1
(43) Pub. Date: Aug. 3, 2023

(54) BRAIN ACTIVITY SYNCHRONIZATION

(71) Applicant: NeuroIndex Ltd., Yokneam (IL)

(72) Inventor: Goded SHAHAF, Haifa (IL)

(73) Assignee: NeuroIndex Ltd., Yokneam (IL)

(21) Appl. No.: 17/421,451

(22) PCT Filed: Jan. 8, 2020
(86) PCT No.: PCT/IL2020/050033

§ 371 Date: Jul. 8, 2021

Related U.S. Application Data
(60) Provisional application No. 62/789,915, filed on Jan. 8, 2019.

(51) Int. Cl. A61B 5/369; A61N 1/04

(52) U.S. Cl. CPC A61B 5/369; A61N 1/04; A61N 1/36014

(57) ABSTRACT

A method for stroke evaluation, including:
recording electrical signals from a first anterior location and from a second anterior location on a subject head, wherein each of the first anterior location and the second anterior location are above different brain hemispheres;
estimating synchronization of brain activity based on a correlation between the recorded electrical signals;
evaluating stroke or at least one parameter of stroke in the subject based on the estimated synchronization.

### RELATED APPLICATIONS

This application claims the benefit of priority under 35 USC § 119(e) of U.S. Provisional Patent Application No. 62/789,915 filed 8 Jan. 2019, the contents of which are incorporated herein by reference in their entirety.

This application is a continuation in part (CIP) of application PCT/IB2018/001632 having an international publication number WO2019/145748A2, filed 5 Dec. 2018.

The contents of the above application are incorporated by reference as if fully set forth herein in their entirety.

### FIELD AND BACKGROUND OF THE INVENTION

The present invention, in some embodiments thereof, relates to evaluating brain function and/or brain health and, more particularly, but not exclusively, to evaluating brain function and/or brain health optionally using an estimate of brain synchronization.

Acute stroke under anesthesia is a dreadful complication, with a non-negligible prevalence, especially in susceptible clinical populations and certain types of operations. Importantly, over recent years there has been significant improvement in the ability to treat acute stroke with advances in thrombolytic pharmacological treatment and mechanical thrombectomy. Rapid identification of acute stroke and/or a penumbra, for example under anesthesia, might lead to crucial and effective intervention.

### SUMMARY OF THE INVENTION

### Some Examples of Some Embodiments of the Invention are Listed Below:

Example 1. A method for stroke evaluation, comprising:
recording electrical signals from a first anterior location and from a second anterior location on a subject head, wherein each of said first anterior location and said second anterior location are above different brain hemispheres;
estimating synchronization of brain activity based on a correlation between said recorded electrical signals;
evaluating stroke or at least one parameter of stroke in said subject based on said estimated synchronization.
Example 2. A method according to example 1, comprising:
delivering a stimulation configured to evoke brain activity in said subject prior to and/or during said recording.
Example 3. A method according to any one of the previous examples, comprising:
filtering said recorded electrical signals to include signals with frequency values in a frequency range of 1-4 Hz, and wherein said estimating comprises estimating synchronization of brain activity based on said electrical signals with frequency values of 1-4 Hz.
Example 4. A method according to any one of the previous examples, wherein said at least one stroke parameter comprises estimated location of necrotic core and/or duration from stroke occurrence.
Example 5. A method according to any one of the previous examples, comprising:
initiating a pharmaceutical treatment and/or a surgical procedure configured to treat said detected stroke in said subject.
Example 6. A method according to any one of the previous examples comprising:
anesthetizing said subject prior to and/or during said estimating.
Example 7. A method according to example 6, comprising:
modifying or stopping said anesthetizing according to said evaluation results.
Example 8. A method according to any one of the previous examples, wherein said recorded electrical signals are EEG electrical signals.
Example 9. A method according to any one of the previous examples, wherein said first anterior location and said second anterior location are above the frontal lobe of the brain.
Example 10. A method according to any one of the previous examples, wherein said first location is location F3 or any location in a distance of up to 10 cm from location F3 and said second location is location F4 or any location in a distance of up to 10 cm from location F4, of an international 10-20 coordinate system.
Example 11. A method for penumbra evaluation, comprising:
recording electrical signals from a first anterior location and from a second posterior location on a subject head, wherein each of said first anterior location and said second posterior location are above different brain hemispheres;
estimating synchronization of brain activity based on a correlation between said recorded electrical signals;
evaluating a penumbra or at least one parameter of penumbra in said subject based on said estimated synchronization.
Example 12. A method according to example 11, comprising:
delivering a stimulation configured to evoke brain activity in said subject prior to and/or during said recording.
Example 13. A method according to any one of examples 11 and 12, comprising:
filtering said recorded electrical signals to include signals with frequency values in a frequency range of 1-4 Hz, and wherein said estimating comprises estimating synchronization of brain activity based on said electrical signals with frequency values of 1-4 Hz.
Example 14. A method according to any one of examples 11 to 13, wherein said evaluating comprises evaluating a global penumbra or at least one parameter of a global penumbra in said subject based on said estimated synchronization.
Example 15. A method according to any one of examples 11 to 14, wherein said at least one penumbra parameter comprises at least one of penumbra size and/or penumbra location.
Example 16. A method according to any one of examples 11 to 15, comprising:
initiating a pharmaceutical treatment and/or a surgical procedure configured to treat said evaluated penumbra in said subject.
Example 17. A method according to any one of examples 11 to 16 comprising:
anesthetizing said subject prior to and/or during said estimating.
Example 18. A method according to example 17, comprising:
modifying or stopping said anesthetizing according to said evaluation results.
Example 19. A method according to any one of examples 11 to 18, wherein said recorded electrical signals are EEG electrical signals.
Example 20. A method according to any one of examples 11 to 19, wherein said first anterior location is above the frontal lobe of the brain, and said second posterior location is above one or more of the parietal lobe, the occipital lobe and/or the temporal lobe of the brain or in between.
Example 21. A method according to any one of examples 11 to 20, wherein said first location is location F3 or any location in a distance of up to 10 cm from location F3 and said second location is location O2 or any location in a distance of up to 10 cm from location O2 of an international 10-20 coordinate system, or said first location is location F4 or any location in a distance of up to 10 cm from location F4 and said second location is location O1 or any location in a distance of up to 10 cm from location O1 of said international 10-20 coordinate system.
Example 22. A device for estimating synchronization of brain activity, comprising:
at least two EEG electrodes each positioned over a different hemisphere of a subject brain;
an EEG measurements unit connected to said at least two electrodes, and configured to receive EEG electrical signals from said at least two electrodes;
a memory
a control circuitry connected to said EEG measurement unit and configured to estimate a synchronization of brain activity in said subject based on a correlation between said received EEG electrical signals using at least one algorithm, lookup table or indications stored in said memory.
Example 23. A device according to example 22, comprising:
a user interface electrically connected to said control circuitry, wherein said control circuitry is configured to signal said user interface to generate a human detectable indication with information regarding said estimated synchronization.
Example 24. A device according to any one of examples 22 or 23, wherein said control circuitry is configured to filter said received EEG electrical signals to include electrical signals with frequency values of 1-4 Hz, and to estimate a synchronization of said brain activity using said filtered electrical signals that have frequency values of 1-4 Hz.
Example 25. A device according to any one of examples 22 to 24, wherein said at least two EEG electrodes are positioned at an anterior location over a frontal lobe of the brain, and wherein said control circuitry is configured to detect stroke or at least one parameter of a stroke based on said estimated synchronization of brain activity.
Example 26. A device according to example 25, wherein said control circuitry is configured to calculate a score indicating an occurrence of stroke in said subject based on said estimated synchronization using at least one algorithm, lookup table or indications stored in said memory.
Example 27. A device according to any one of examples 22 to 24, wherein at least one EEG electrode of said at least two is positioned at an anterior location over the brain, and at least one second electrode of said at least two electrodes is positioned at posterior location over the brain, and wherein said control circuitry is configured to detect a penumbra or a global penumbra in said subject based on said estimated synchronization.
Example 28. A device according to example 27, wherein said control circuitry is configured to calculate a score indicating a global penumbra in said subject based on said estimated synchronization, using at least one algorithm, lookup table or indications stored in said memory.
Example 29. A method for estimating synchronization of brain activity, comprising: recording electrical signals from a first location and from a second location on a subject head, wherein each of said first location and said second location are above different brain hemispheres; estimating synchronization of brain activity based on said recorded electrical signals.
Example 30. A method according to example 29, comprising:
delivering a stimulation configured to evoke brain activity in said subject prior to and/or during said recording.
Example 31. A method according to any one of examples 29 or 30, comprising:
filtering said recorded electrical signals to include signals with frequency values in a frequency range of 1-4 Hz, and wherein said estimating comprises estimating synchronization of brain activity based on said electrical signals with frequency values of 1-4 Hz.
Example 32. A method according to any one of examples 29 to 31, comprising:
identifying recoverable brain tissue in said subject based on said estimated synchronization.
Example 33. A method according to example 32, comprising:
selecting a treatment for said subject according to the results of said identifying.
Example 34. A method according to example 33, wherein said treatment comprises thrombolytic pharmacological treatment or mechanical thrombectomy.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as determining synchronization of brain activity, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a general flow chart of a process for estimating at least one parameter of an ischemic penumbra, according to some exemplary embodiments of the invention;

FIG. 2A is a schematic illustration showing measuring activity of different parts of a brain, according to some exemplary embodiments of the invention;

FIG. 2B is a schematic illustration showing potential locations on a scalp of a subject for positioning electrodes, according to some exemplary embodiments of the invention;

FIG. 3 is a schematic block diagram of a device for measuring activity of a brain, according to some exemplary embodiments of the invention;

FIG. 4A is a flow chart of a process for estimating at least one parameter of a penumbra, for example in a sedated and/or an anesthetized subject, according to some exemplary embodiments of the invention;

FIG. 4B is a flow chart of a process for estimating a stroke event, for example in a sedated and/or an anesthetized subject, according to some exemplary embodiments of the invention;

FIG. 4C is a flow chart of actions performed by a device during estimation of stroke and/or penumbra, according to some exemplary embodiments of the invention;

FIG. 5A is a flow chart showing EEG signal processing, according to some exemplary embodiments of the invention;

FIG. 5B is a flow chart of a process for generation of an anterior synchronization index (ASI), according to some exemplary embodiments of the invention;

FIG. 5C is a flow chart of a process for generation of a global synchronization index (GSI), according to some exemplary embodiments of the invention;

FIG. 6A is a schematic illustration showing a flow of a first validation experiment, and of a method according to some exemplary embodiments of the invention;

FIG. 6B is a graph showing changes in an ASI under anesthesia and of a method according to some exemplary embodiments of the invention;

FIG. 6C is a schematic illustration showing locations on a head of a subject for positioning electrodes for generation of ASI, as performed in the first validation experiment and according to some exemplary embodiments of the invention;

FIG. 7A is a graph showing pre procedural NIHSS values of subject participating in the first validation experiment, grouped by post procedural outcome;

FIG. 7B is a graph showing pre procedural ASI values of subject participating in the first validation experiment, grouped by post procedural outcome;

FIG. 8A is a graph showing changes in a global synchronization index under anesthesia;

FIG. 8B is a schematic illustration showing locations on a head of a subject for positioning electrodes for generation of GSI, as performed in the first validation experiment and according to some exemplary embodiments of the invention;

FIG. 9A is a graph showing changes in ASI between different groups of a second validation experiment;

FIG. 9B is a graph showing changes in ASI before and after catheterization; and

FIGS. 10A-10C are graphs showing a relation between GSI and ASI in different groups of the second validation experiment.

### DESCRIPTION OF SPECIFIC EMBODIMENTS OF THE INVENTION

The present invention, in some embodiments thereof, relates to evaluating brain function and/or brain health and, more particularly, but not exclusively, to evaluating brain function and/or brain health optionally using an estimate of brain synchronization.

An aspect of some embodiments relates to estimating at least one parameter of a penumbra based on a relation between activities of two or more brain regions. In some embodiments, the at least two brain regions are brain regions located on different hemispheres. In some embodiments, the two or more brain regions comprise at least one anterior brain region and at least one posterior brain region, each located at different hemispheres of the brain. As used herein, an anterior position is a position over the frontal lobe of the brain. As used herein, a posterior position is a position over parietal, occipital or temporal lobes of the brain (or among them). As used herein, a brain region is a region of a brain having a volume of up to 50% of a total volume of a brain. As used herein, a brain region is a region of a brain is selected from one or more of frontal lobe, parietal, occipital or temporal lobes, or any combination or sub-region thereof. In some embodiments, the relation between the two or more brain regions comprise level of synchronization, for example synchronization in at least one of, time, amplitude and frequency of electrical signals recorded from the two or more brain regions. In some embodiments, the relation between activity of the at least one anterior brain region and the activity of at least one posterior brain region of different hemispheres, is used to generate a global synchronization index (GSI), also termed herein as a global synchronization (GS).

According to some embodiments, the at least one parameter of penumbra comprises at least one of a presence of a penumbra, penumbra size, and/or penumbra location. Additionally or alternatively, the at least one parameter of the penumbra comprises a stage of the penumbra or characterization of the penumbra tissue. In some embodiments, characterization of penumbra tissue comprises estimating whether cells in the penumbra are similar to cells in brain tissues not affected by the ischemic event or that the cells in the penumbra are more similar to cells in an ischemic core in the brain. In some embodiments, the at least one parameter of the penumbra comprises a probability of tissue in the penumbra to be rescued, for example if performing specific therapeutic procedures, for example mechanical thrombectomy and pharmacological thrombolysis. In some embodiments, a probability of tissue in the penumbra to be rescued is correlated with at least one treatment or a best practice table.

According to some embodiments, the at least one parameter of the penumbra is estimated based on EEG signals recorded by at least 2, 3, 4, 5, 6 or any intermediate, smaller or larger number of EEG electrodes positioned in at least one anterior location, and in at least one posterior location, for example above on a scalp of a subject, each above a different hemisphere of a brain of the subject. Alternatively, the at least one parameter of the penumbra is estimated based on electrical signals recorded by a single electrode, for example a bipolar electrode or use a squid or other magnetic sensor to measure local activity, that is moved between the at least one anterior location and at least one posterior location on the scalp. Optionally, the at least one parameter of the penumbra is estimated based on the recorded EEG signals and an additional measurement, for example MRI, CT, NIRS, PET/SPECT, US-Doppler, MEG.

According to some embodiments, the EEG signals are recorded in a timed relation, for example prior to and/or during a stimulation delivered to the subject. In some embodiments, the delivered stimulation, for example an auditory, a visual, or any other sensory stimulation is configured to evoke a response, for example a brain response in the subject.

According to some embodiments, the at least one parameter of the penumbra is estimated while the subject is awake, for example when the subject is hospitalized in a hospital, in the subject home or in any hospitalization facility. In some embodiments, the at least one parameter of the penumbra is estimated, for example by a caregiver or an early responder, for example, a nurse, a physician, and a paramedic, outside a hospital. In some embodiments, the at least one parameter of the penumbra is estimated in an emergency room, for example as part of a triage process. Alternatively, the at least one parameter of the penumbra is estimated while the subject is sedated and/or anesthetized, for example during a surgical procedure. Optionally, the surgical procedure and/or anesthesia delivery is modified based on the results of the penumbra parameter assessment.

An aspect of some embodiments relates to estimating at least one parameter of a stroke event, for example an acute stroke event, in a subject based on a relation between activities of two or more brain regions or areas of the brain. In some embodiments, the two or more brain regions comprise anterior brain regions, each located at different hemispheres of the brain. In some embodiments, the relation between the two or more brain regions comprise level of activities synchronization, for example synchronization in at least one of, time, amplitude and frequency of electrical signals recorded from the two or more brain regions. In some embodiments, the relation between the two anterior brain regions is used to generate an anterior synchronization index (ASI), also termed herein as an interhemispheric synchronization (IS).

According to some embodiments, the at least one parameter of the stroke event comprises an occurrence of a stroke event, a location of an ischemic core in the brain and/or a size of the ischemic core. Additionally, the at least one parameter of the stroke event comprises a time or a time window in which the stroke event has occurred, or an estimation of the time that passed between the occurrence of the stroke event and the beginning of the stroke event estimation process.

According to some embodiments, the at least one parameter of the stroke event is estimated based on EEG signals recorded by at least 2 EEG electrodes positioned in anterior locations on a scalp of a subject, where each of the anterior locations is above a different hemisphere of a brain of the subject. Alternatively, the at least one parameter of the stroke event is estimated based on EEG signals recorded by a single electrode, that is moved between the two anterior locations. Optionally, the at least one parameter of the stroke is estimated based on the recorded EEG signals and an additional measurement, for example MRI, CT, NIRS, PET/SPECT, US-Doppler, MEG.

According to some embodiments, the EEG signals are recorded in a timed relation, for example prior to and/or during a stimulation delivered to the subject, for example as described above.

According to some embodiments, the at least one parameter of the stroke is estimated while the subject is awake, for example when the subject is hospitalized in a hospital, in the subject home or in any hospitalization facility. In some embodiments, the at least one parameter of the stroke is estimated, for example by a caregiver or an early responder, for example, a nurse, a physician, and a paramedic, outside a hospital. In some embodiments, the at least one parameter of the stroke is estimated in an emergency room, for example as part of a triage process. Alternatively, the at least one parameter of the stroke is estimated while the subject is sedated and/or anesthetized, for example during a surgical procedure. Optionally, the surgical procedure and/or anesthesia delivery is modified based on the results of the stroke parameter assessment.

An aspect of some embodiments relates to estimating at least one parameter of salvageable brain tissue based on brain activity synchronization. In some embodiments, the at least one parameter of the salvageable brain tissue is estimated based on a relation between activities of two or more brain regions or areas of the brain. In some embodiments, the at least one parameter comprises size of salvageable tissue, location of salvageable tissue, probability of salvageable tissue to respond to a specific treatment, for example thrombectomy or a pharmaceutical treatment.

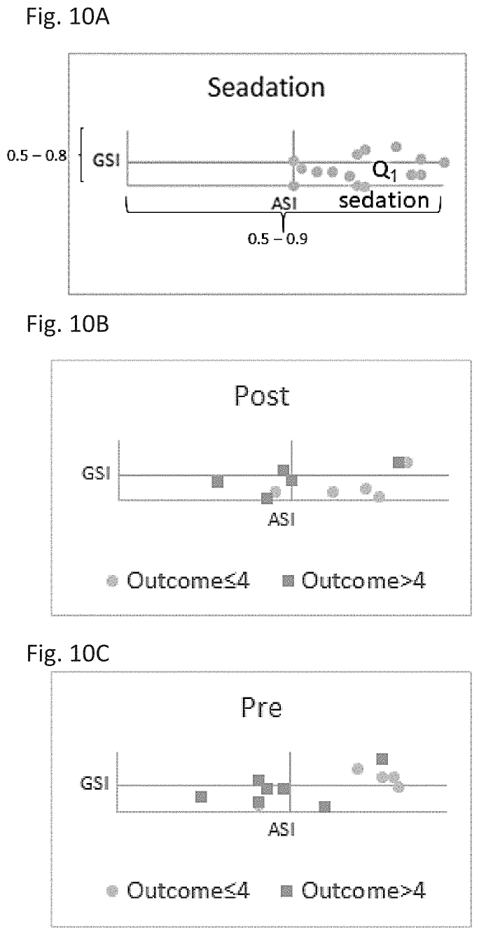

According to some embodiments, a matrix, for example a table or an algorithm includes a relation between one or more brain activity synchronization indices, and a clinical condition or a probability of a clinical condition, for example an acute stroke, penumbra, and global penumbra. In some embodiments, the information in the matrix indicates a probability of a subject to have one or more of the clinical conditions. In some embodiments, a treatment is selected using the matrix. In some embodiments, an example of a matrix is shown in FIGS. 10A-10C.

According to some embodiments, the matrix is generated by measuring synchronization in a plurality of patients prior to a treatment, for example thrombectomy, and monitoring an outcome of the patients following the treatment.

According to some embodiments, as described herein, electrical signals, for example EEG signals, are recorded from a subject. In some embodiments, the electrical signals are recorded from at least two locations, each above a different brain hemisphere. In some embodiments, out of the recorded electrical signals, electrical signals having low frequencies, for example frequencies in a range of 1-4 Hz, are used to determine a level of correlation between signals recorded from the at least two locations. Optionally, the correlation is determined after removal of noisy segments and/or epoch of the recorded signals.

According to some embodiments, the determined correlation indicates a level of synchronization between recorded electrical signals from the at least two locations, and is used to generate one or more indices, for example the ASI and the GSI. In some embodiments, the level of synchronization is divided to low synchronization level, medium synchronization level, high synchronization level and very high synchronization level.

In some embodiments, electrical signals are recorded from one or more electrodes, configured to measure brain activity, for example EEG electrodes.

In some embodiments, the recorded electrical signals are filtered to include signals, for example EEG signals with frequency in a range of 1-6 Hz, for example 1-5 Hz, 1-4 Hz, 1-3 Hz or any intermediate, smaller or larger range of frequencies.

In some embodiments, the filtered signals are divided into segments having a similar or varying length. In some embodiments, a duration of each segment is in a range of 1-120 seconds, for example 1-10 seconds, 5-25 seconds, 10-50 seconds or any intermediate, shorter or longer duration.

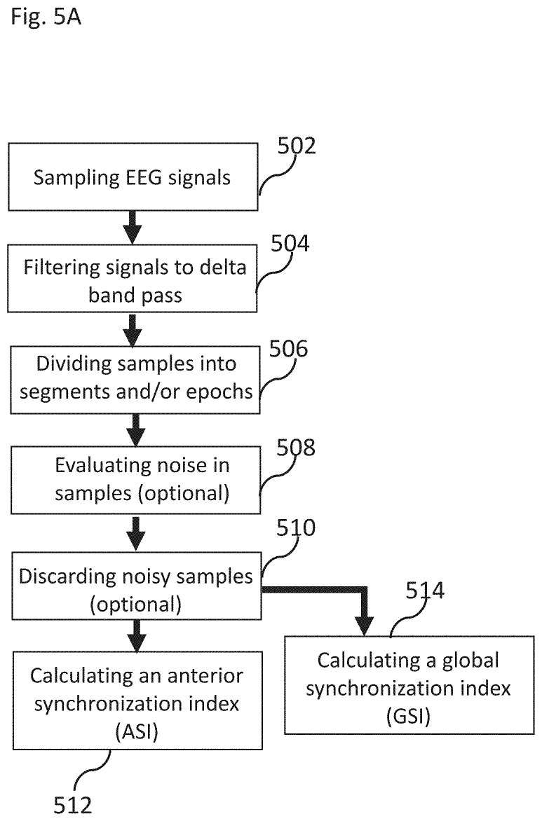

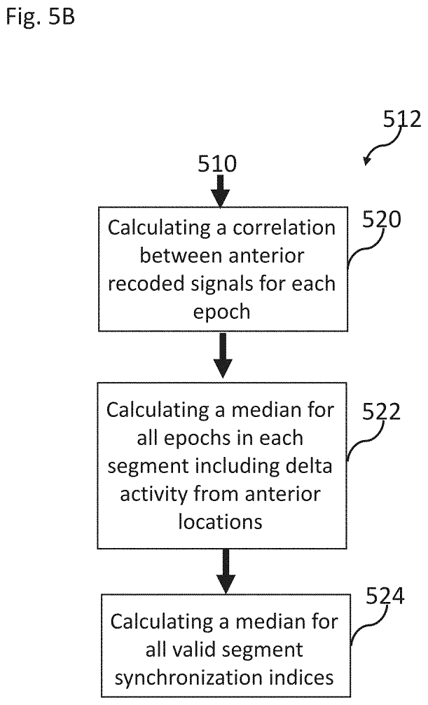

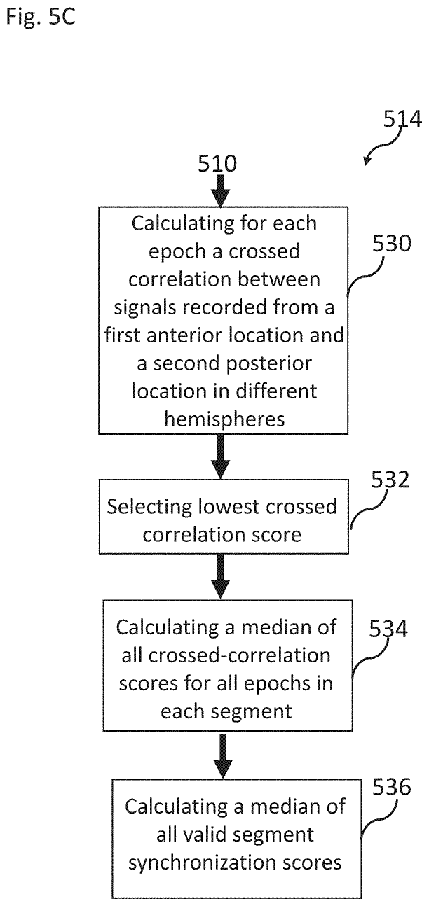

In some embodiments, noisy segments are discarded. In some embodiments, noisy segments are segments that include noise which is higher than a predetermined value, for example as described in the exemplary experiment II, and in FIG. 5A.

In some embodiments, undiscarded segments are divided into epochs. In some embodiments, a duration of each epoch is in a range of 1-120 seconds, for example 1-10 seconds, 5-25 seconds, 10-50 seconds or any intermediate, shorter or longer duration.

In some embodiments, a correlation between different epochs is evaluated. In some embodiments, statistical methods and or algorithms are applied on the evaluated correlations, for example to identify epochs with a desired correlation. In some embodiments, the statistical methods include a median, a mean, an average or a weighted average.

In some embodiments, a synchronization index, for example ASI and GSI is generated based on a median correlation of at least some or all segments having epochs with the desired correlation. In some embodiments, a synchronization index is a median correlation of correlations or a median of the correlations of at least some or all segments having epochs with the desired correlation. In some embodiments the generated synchronization index is at least 20% linear over at least 80% of the index range. In some embodiments, any other correlation index that is at least 20% linear over at least 80% of the index range can be used to estimate, calculate, and/or determine a correlation between activity of 2 or more brain regions or brain areas.

Any method, process or device described in PCT/IB2018/001632 (WO2019/145748A2) can be used in one or more of the embodiments described in this application.

A potential advantage of estimating stroke and penumbra parameters may be to deliver an urgent and effective treatment to a subject in order to rescue as much brain tissue as possible following a stroke event, for example while the subject is sedated and/or anesthetized.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary General Process for Estimating a Parameter of a Penumbra

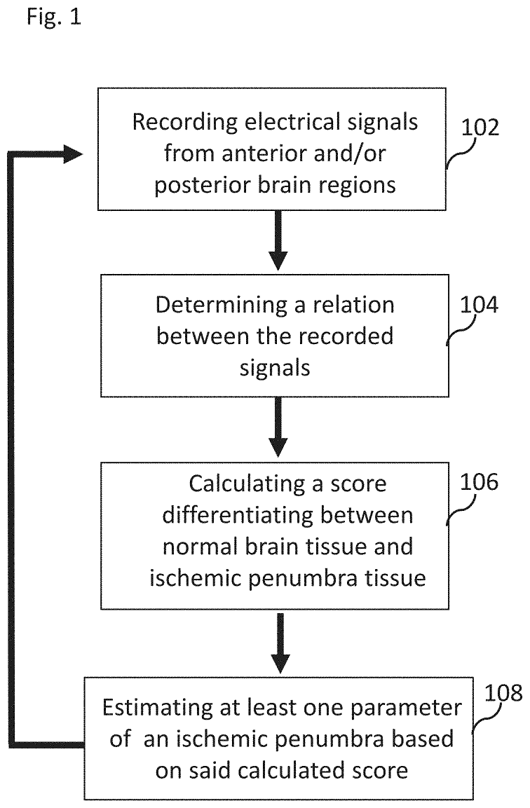

According to some exemplary embodiments, a penumbra, for example an ischemic penumbra, is estimated, for example to determine if and/or how brain tissue in the penumbra can be rescued following an ischemic event. In some embodiments, at least one parameter of the penumbra, for example a presence of a penumbra, penumbra size and/or location is estimated while a subject is sedated and/or anesthetized. Reference is now made to FIG. 1 depicting a general process for estimating at least one parameter of a penumbra, for example an ischemic penumbra, according to some exemplary embodiments of the invention.

According to some exemplary embodiments, electrical signals are recorded from anterior and/or posterior brain regions at block 102. In some embodiments, the electrical signals are recorded from at least one anterior brain region, and from at least one posterior brain region, each located at a different hemisphere of a brain. In some embodiments, the electrical signals are recorded by at least two electrodes, each positioned on a scalp of a subject above a different brain region selected from the at least one anterior brain region and the at least one posterior brain region. Alternatively, a single electrode, for example an EEG electrode, is moved on a scalp of a subject between at least two measurement locations, a first measurement location above the anterior brain region, and a second measurement location above the posterior brain region.

According to some exemplary embodiments, the electrical signals are recorded in a timed relation to a stimulation delivered to the subject, for example following and/or during the stimulation delivery. In some embodiments, the delivered stimulation is configured to induce an evoked response in the brain if the subject, for example in the at least one anterior brain region and/or the at least one posterior brain regions, or in neural circuits affecting one or both of these regions.

According to some exemplary embodiments, a relation between the recorded signals is determined at block 104. In some embodiments, a relation is determined between electrical signals measured from the at least one anterior brain region, and electrical signals measured from the at least one posterior brain region. In some embodiments, the determined relation comprises one or more of, relation between timing and/or duration of the recorded signals or selected segments within the recorded signals, and/or relation between amplitude and/or frequency of the recorded signals or in selected segments within the recorded signals.

According to some exemplary embodiments, a score is calculated based on the determined relation, at block 106. In some embodiments, the calculated score, comprises a GSI score, configured to differentiate between healthy brain tissue, for example brain tissue not affected by an ischemic event, and a penumbra, for example an ischemic penumbra tissue.

According to some exemplary embodiments, at least one parameter of the penumbra is estimated at block 108 based on the calculated score, at block 108. In some embodiments, the at least one parameter comprises at least one of size, location, expansion rate of the penumbra, and/or penumbra stage, for example whether functionality of the cells in the penumbra is closer to functionality of cells in healthy brain tissue or to functionality of cells in an ischemic core in the brain.

According to some exemplary embodiments, the process or recording, determining, calculating and estimating is continuously repeated, for example to monitor changes in the estimated parameter over time. In some embodiments, the process is repeated for a time period of at least 10 seconds, for example at least 30 seconds, at least 1 minute, at least 30 minutes, at least 1 hour, at least 10 hours, or any intermediate, smaller or larger time duration. In some embodiments, the process is repeated as long as a patient is anesthetized during a surgical procedure, or as long as a surgical procedure is performed. Alternatively, the process is performed intermittently or on-demand.

Exemplary Recording Activity from Brain Regions

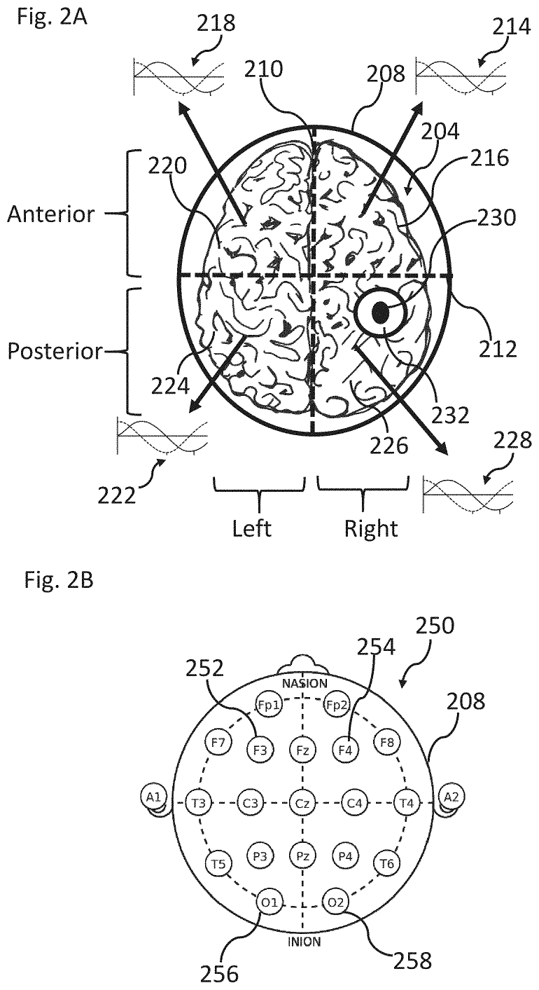

According to some exemplary embodiments, activities of two or more brain regions, for example 2, 3, 6 or any intermediate, smaller or larger number of brain regions, are recorded. In some embodiments, the activity of the brain regions is recorded by at least one electrode, for example by placing at least one electrode, on a scalp of a subject and moving the electrode between different locations of the scalp. Alternatively, the activities of the two or more brain regions are recorded by two or more electrodes positioned at different locations on the scalp. Reference is now made to FIG. 2A, depicting recording electrical signals from different regions of a brain, according to some exemplary embodiments of the invention.

According to some exemplary embodiments, a brain 204 is positioned inside a skull 208. A sagittal axis 210 of the brain 204 divides the brain 204 into two hemispheres, a left hemisphere and a right hemisphere. Additionally, a coronal axis 212 of the brain 204 divides the brain 204 into an anterior portion and a posterior portion. As used herein, an anterior position is a position over the frontal lobe of the brain. As used herein, a posterior position is a position over parietal, occipital or temporal lobes of the brain (or among them).

According to some exemplary embodiments, placing an electrode, for example, an EEG electrode above an anterior left hemisphere brain region 220, allows to record electrical activity 218 from brain region 220. In some embodiments, placing an electrode, for example, an EEG electrode above an anterior right hemisphere brain region 216, allows to record electrical activity 214 from brain region 216. In some embodiments, placing an electrode, for example, an EEG electrode above a posterior left hemisphere brain region 224, allows to record electrical activity 222 from brain region 222. In some embodiments, placing an electrode, for example, an EEG electrode above a posterior right hemisphere brain region 226, allows to record electrical activity 228 from brain region 226.

According to some exemplary embodiments, a relation between recorded activities of different brain regions is used to estimate at least one parameter of a stroke event, for example a location and/or size of a necrotic core 230. Alternatively or additionally, the relation between the recorded activities is used to estimate at least one parameter related to a penumbra 232, which optionally surrounds the necrotic core 230.

According to some exemplary embodiments, an anterior synchronization index, for example ASI, is generated based on a relation between activity recorded from an anterior region of the first hemisphere and activity recorded from an anterior region of the second hemisphere, for example activities recorded from brain regions 220 and 216. In some embodiments, a global synchronization index, for example GSI, is generated based on a relation between activity recorded from an anterior brain region of a first hemisphere and activity recorded from a posterior brain region of the second hemisphere, for example activities recorded from brain regions 220 and 226, or from brain regions 216 and 224.

According to some exemplary embodiments, at least 4 electrodes are positioned on a scalp of a patient, each electrode over a different brain region selected from brain regions 220, 216, 224, and 226. In some embodiments, at least two electrodes are positioned over anterior regions of the brain, for example over brain region 220 and over brain region 216. In some embodiments, additional electrodes are positioned, for example to have more electrodes per brain region, for example to increase spatial resolution of the EEG signal. Optionally, increasing the number of electrodes per brain region and/or determining a relation between activities of different anterior and/or posterior brain regions, allows, for example, to increase an estimation accuracy for detecting a stroke and/or a penumbra, or parameters thereof.

Reference is now made to FIG. 2B, depicting optional locations for positioning of electrodes, for example EEG electrodes, according to some exemplary embodiments of the invention.

According to some exemplary embodiments, a map 250 of the scalp comprises a plurality of electrode positioning locations on a scalp of a human subject. In some embodiments, position 252 located above an anterior left hemisphere brain region, for example brain region 220 shown in FIG. 2A, and position 254 located above an anterior right hemisphere brain region, for example brain region 216 shown in FIG. 2A, are used for placement of electrodes to generate an ASI. In some embodiments, any position over the anterior left hemisphere of the brain can be used with any position over the anterior right hemisphere of the brain, for generating an ASI or GSI, or to determine any relation between an activity of anterior brain regions of both hemispheres.

According to some exemplary embodiments, position 256 over a posterior left brain region, and position 258 over the right posterior brain region, together with positions 252 and 254 are used to generate a GSI. Alternatively, any other position on the scalp over the right posterior brain region can be used with any position on the scalp over the left anterior brain region, for generating the GSI. Alternatively or additionally, any other position on the scalp over the left posterior brain region can be used with any position on the scalp over the right anterior brain region, for generating the GSI.

### Exemplary Device

According to some exemplary embodiments, a device for estimating stroke and/or penumbra or parameters thereof, is used in surgical procedures, for example to monitor a clinical condition of a patient under anesthesia. In some embodiments a device for estimating stroke and/or penumbra or parameters thereof, is used by early responders or in a hospital, for example during a triage procedure. In some embodiments a device for estimating stroke and/or penumbra or parameters thereof, is used to monitor a clinical condition of a subject, for example a hospitalized subject in a hospital, at home or in any hospitalization facility. In some embodiments, the device is used to monitor a clinical condition in a risk to develop stroke, for example surgical operations, especially operations that require a halt or reduction of protective treatment (e.g. antiaggregant or anti-coagulant); hypercoagulation states; following previous stroke or TIA; higher risk according to known scales, such as the ABCD2 scale.

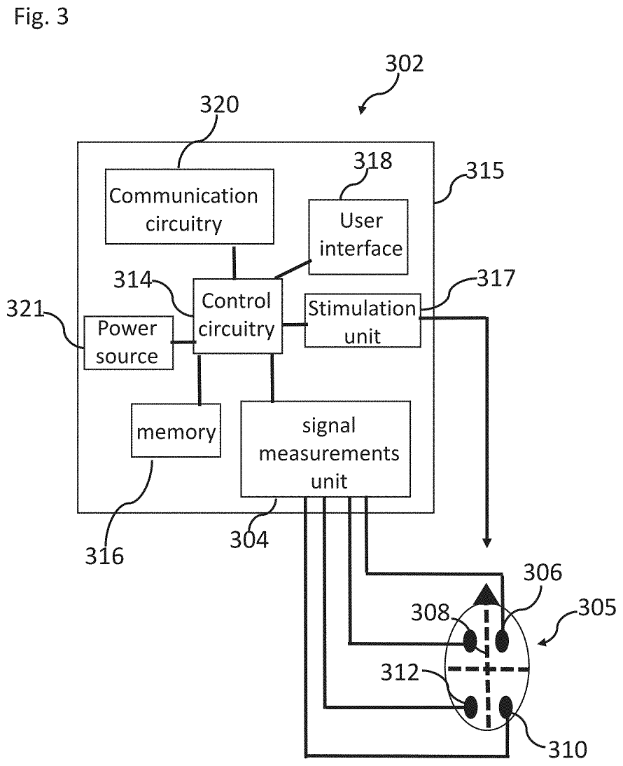

According to some exemplary embodiments, a device for estimating stroke and/or penumbra, or parameters thereof, is movable and is optionally wearable, for example to allow easy and continuous monitoring of a subject clinical condition. Reference is now made to FIG. 3, depicting a device for estimating stroke and/or penumbra or parameters thereof, according to some exemplary embodiments of the invention.

According to some exemplary embodiments, a device for estimating stroke and/or or penumbra, for example device 302 comprises a signal measurement unit, for example EEG measurements unit 304 connectable to at least one electrode for example an EEG electrode. In some embodiments, the unit 304 is connected, for example electrically connected, to at least one EEG electrode, for example EEG electrodes 306, 308, 310, 312. In some embodiments, the at least one EEG electrode is attached to a scalp of a subject 305. In some embodiments, EEG electrode 308 is attached to the scalp 305 above an anterior brain region of the left hemisphere. In some embodiments, EEG electrode 306 is attached to the scalp 305 of the subject above an anterior brain region of the right hemisphere. In some embodiments, EEG electrode 310 is attached to the scalp 305 above a posterior brain region of the right hemisphere. In some embodiments, EEG electrode 312 is attached to the scalp 305 above a posterior brain region of the right hemisphere.

According to some exemplary embodiments, the at least one EEG electrode, for example EEG electrodes 06, 308, 310 and 312, comprises EEG electrodes of any type, for example wet EEG electrodes, dry EEG electrodes, active EEG electrodes, passive EEG electrodes or any combination thereof.

According to some exemplary embodiments, the unit 304 is configured to receive electrical signals from two or more channels, for example from the two anterior electrodes 306 and 308, each positioned over a different hemisphere, when the device 302 is used to calculate an anterior index, for example a score of ASI. Alternatively, the unit is configured to receive electrical signals from four or more channels, for example from EEG electrodes 306, 308, 310 and 312, when the device 302 is used to calculate a global index, for example a score of GSI. In some embodiments, the signal measurements unit is connectable to any type of an electrode or sensor configured to record activity of anterior and/or posterior brain regions, for example Magnetoencephalography (MEG) electrodes, Near Infrared Spectroscopy (NIRS) electrodes, or any combination between these electrodes and EEG electrodes.

According to some exemplary embodiments, the device 302 comprises a control circuitry 314 electrically connected to the unit 304. In some embodiments, the device 302 comprises memory 316, electrically connected to the control circuitry 314. In some embodiments, signal measurements received by unit 304 are transmitted to control circuitry 314, and are stored in memory 316.

According to some exemplary embodiments, the device 302 comprises a stimulation unit, for example stimulation unit 317, electrically connected to the control circuitry 314. In some embodiments, the stimulation unit 317 is configured to deliver a stimulation, or example an audio and/or a visual stimulation to a subject from which brain activity is recorded. In some embodiments, the stimulation is configured to evoke a response, for example a brain response in the subject. In some embodiments, the control circuitry 314 is configured to synchronize a delivery of the stimulation by the stimulation unit 317 with the recording of brain activity by the electrodes. In some embodiments, the control circuitry 314 signals the stimulation unit 317 to generate and deliver the stimulation in a timed relation, for example prior to and/or during the recording of the brain activity.

According to some exemplary embodiments, the control circuitry 314 is configured to filter and/or to process at least some of the electrical signals stored in the memory 316 or the electrical signals received from the unit 304, using one or more algorithms and/or programs stored in the memory 316. In some embodiments, the control circuitry 314 is configured to process signals received by one or more, for example 2,3,4 or any intermediate, smaller or larger number of channels, for example channels from electrodes recording brain activity of the subject. In some embodiments, the control circuitry 314 is configured to calculate one or more synchronization indices, for example ASI and/or GSI based on electrical signals received from two or more channels from electrodes recording brain activity of the subject. In some embodiments, the control circuitry 314 is configured to calculate the one or more synchronization indices using at least one algorithm of program stored in the memory 316. In some embodiments, algorithms used to calculate synchronization indices comprise, for example, time-based algorithms or frequency-based algorithms.

According to some exemplary embodiments, the device 302 comprises a user interface, for example user interface 318, configured to generate and deliver an indication, for example a human detectable indication to a user of the device 302, for example a technician, a caregiver, or an expert monitoring the activity of the device, and/or the condition of the subject. In some embodiments, the user interface 318 comprises a speaker or a display for delivery of the indication. In some embodiments, the control circuitry 314 signals the user interface 318 to generate and deliver the indication. Additionally, the user interface 318 comprises at least one input unit, for example a keyboard, a button, a selection knob, a computer mouse, configured to receive input signals from a user of the device 302. In some embodiments, the at least one input unit is configured to insert personal details of a subject and/or to select or modify index measurement protocols or parameters thereof. In some embodiments, the at least one input unit is configured to allow selection of a specific index for calculation, for example an ASI and/or a GSI.

According to some exemplary embodiments, the device 302 comprises a communication circuitry, for example communication circuitry 320 configured to generate and deliver signals to devices located in the vicinity of the device 302, for example anesthesia device or any other device in a surgical operation room or in an emergency room. Alternatively or additionally, the device 302 is configured to generate and deliver signals, for example wireless signals to a remote device, for example a remote computer, a remote mobile device, a remote cloud storage and/or a remote server. In some embodiments, the control circuitry 314 is configured to signal the communication circuitry to generate and deliver an indication to the remote device or to at least one device located at a vicinity of the device 302, for example based on a synchronization score calculated by the control circuitry 314.

According to some exemplary embodiments, the device 302 is configured to deliver the recorded electrical signals to a remote device, for example to a remote server or to a remote storage cloud, for example via the communication circuitry 320. In some embodiments, the calculation of the synchronization index, for example ASI and/or GSI is performed in the remote device, for example using at least one algorithm or a program stored in the remote device. In some embodiments, a score of the synchronization index or an indication regarding estimation of stroke and/or penumbra is received via the communication circuitry 320 of the device 302.

According to some exemplary embodiments, a casing 315 of the device is small enough and light enough to be wear by the monitored subject. In some embodiments, the casing 315 comprises at least one clip, strap, hook, handle or any other attachment means, configured to allow attachment of the device 302 to a monitored subject, for example to the clothes or body of the monitored subject. In some embodiments, the electrodes, for example the EEG electrodes, are part of a hat or a strap, shaped and sized to be positioned on a head of a subject for time periods longer than 10 minutes, for example time periods of at least 15 minutes, at least 30 minutes, at least one hour, at least 2 hours, at least 10 hours, at least 24 hours, or any intermediate, smaller or larger time duration.

According to some exemplary embodiments, the device 302 comprises a power source 321 electrically connected to the control circuitry. In some embodiments, the power source 321 comprises a battery, for example a rechargeable battery 321.

### Exemplary Detailed Process for Estimating a Penumbra

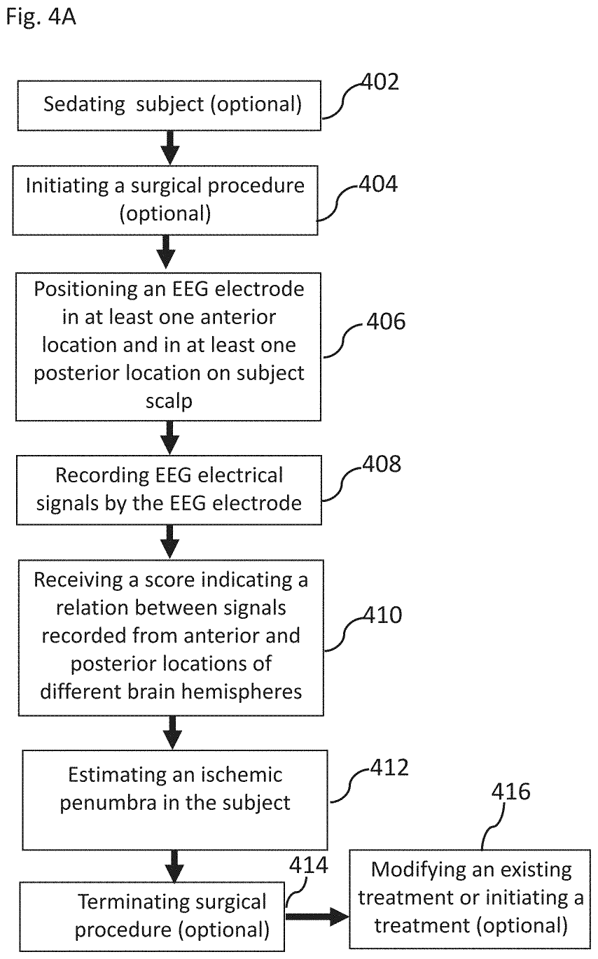

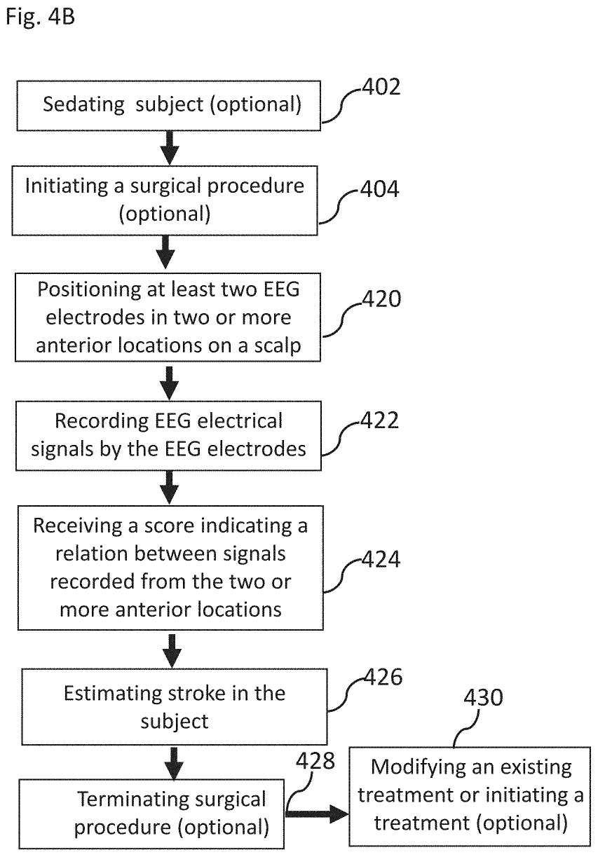

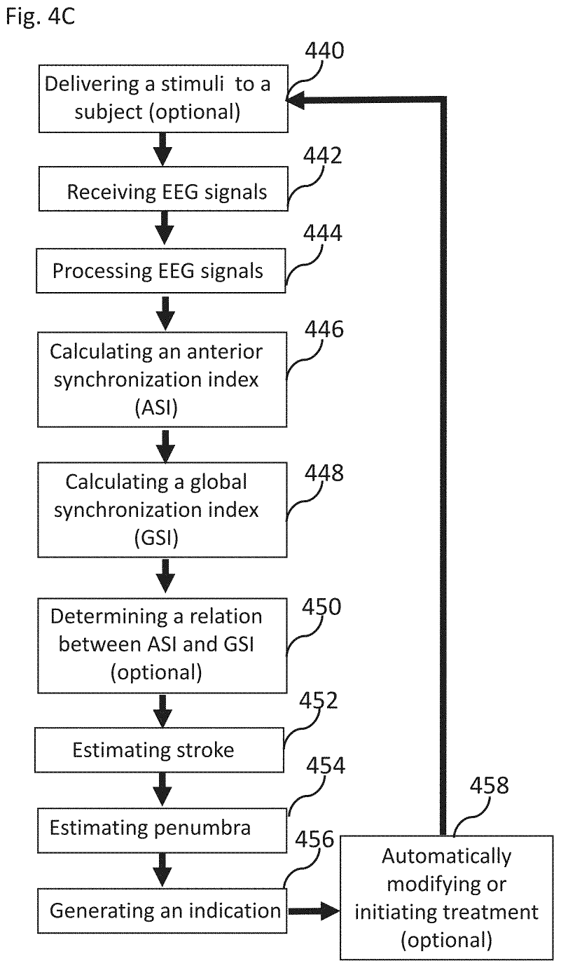

Reference is now made to FIG. 4A, depicting a detailed process for estimating a penumbra, according to some exemplary embodiments of the invention.

According to some exemplary embodiments, a subject is sedated at block 402. In some embodiments, the subject is anesthetized at block 402. In some embodiments, the subject is sedated and/or anesthetized prior to a surgical procedure.

According to some exemplary embodiments, a surgical procedure is initiated at block 404.

According to some exemplary embodiments, at least one EEG electrode, for example 2 electrodes are positioned in at least one anterior location, and in at least one posterior location of a subject scalp, at block 406. In some embodiments, the at least one anterior location and the at least one posterior location on the scalp are above different brain hemispheres. In some embodiments, 4 EEG electrodes are positioned on the scalp, for example as shown in FIGS. 2A, 2B and 3. In some embodiments, the electrodes are positioned prior to the initiation of the surgical procedure at block 404 or prior to the sedation at block 402.

According to some exemplary embodiments, EEG electrical signals are recorded at block 408. In some embodiments, the EEG electrical signals are recorded during or following a stimulation delivered to the subject, for example to evoke a brain response. In some embodiments, the EEG electrical signals are recorded for example simultaneously from two or more electrodes positioned on a scalp of the subject at block 406.

According to some exemplary embodiments, a score indicating a relation between electrical signals recorded from anterior and posterior locations of different brain hemispheres, is received at block 410. In some embodiments, the received score is a score of a GSI. In some embodiments, the received score indicates a relation between electrical signals recorded from two or more, for example 3,4,5,6 locations on the scalp.

According to some exemplary embodiments, an ischemic penumbra or parameters thereof, is estimated in the subject at block 412. In some embodiments, an ischemic penumbra or parameters thereof is estimated based on the score calculated at block 410. In some embodiments, the parameters of the penumbra comprise presence of a penumbra, size and/or location of the penumbra.

According to some exemplary embodiments, the surgical procedure is terminated or modified at block 414. In some embodiments, the surgical procedure is terminated or modified based on the results of the penumbra estimation.

According to some exemplary embodiments, an existing treatment of the subject, is modified at block 416, for example based on the penumbra estimation results. Alternatively or additionally a new treatment of the subject is initiated at block 416 based on the penumbra estimation results.

### Exemplary Detailed Process for Estimating Stroke

Reference is now made to FIG. 4B depicting estimation of stroke, according to some exemplary embodiments of the invention.

According to some exemplary embodiments, a subject is sedated at block 402, and a surgical procedure is optionally initiated at block 404, for example as described with respect to FIG. 4A.

According to some exemplary embodiments, at least two electrodes, for example EEG electrodes, are positioned on a scalp of the subject, at block 420. In some embodiments, each of the at least two electrodes is positioned on the scalp above an anterior brain region of a different hemisphere.

According to some exemplary embodiments, electrical signals, for example EEG electrical signals are recorded at block 422. In some embodiments, the electrical signals are recorded, for example simultaneously, from the at least two electrodes positioned on the scalp of the subject at block 420.

According to some exemplary embodiments, a score indicating a relation between signals recorded from two or more anterior locations on the scalp is received at block 424. In some embodiments, the two or more anterior locations are anterior locations on the scalp above different hemispheres. In some embodiments, the score indication comprises a score of an ASI.

According to some exemplary embodiments, a stroke is estimated in the subject, at block 426, based on the received score. In some embodiments, a stroke or at least one parameter thereof is estimated at block 426. In some embodiments, the at least one parameter comprises estimated location of the stroke or necrotic core, estimated size of the necrotic core and/or time duration from an occurrence of a stroke event.

According to some exemplary embodiments, the surgical procedure initiated at block 404 is terminated at block 428, following the estimation of stroke. Alternatively, the surgical procedure is modified following the estimation of stroke.

According to some exemplary embodiments, an existing treatment of the subject, for example an existing drug regime of the subject, is modified at block 430 following the estimation of stroke. In some embodiments, at least one treatment is initiated in the subject, for example initiating a treatment with blood diluting compounds, following the estimation of stroke.

### Exemplary Device Actions

Reference is now made to FIG. 4C, depicting actions performed by a device, for example device 302 shown in FIG. 3, according to some exemplary embodiments of the invention.

According to some exemplary embodiments, a stimulus is delivered to a subject at block 440. In some embodiments, the stimulus is configured to evoke a brain response in the subject. In some embodiments, the stimulus comprises an audio and/or a visual stimulus. In some embodiments, the stimulus is delivered by stimulation unit 317 shown in FIG. 3.

According to some exemplary embodiments, electrical signals, for example EEG signals, are received at block 442. In some embodiments, the electrical signals are received by a signal measurements unit, for example unit 304 shown in FIG. 3. In some embodiments, the electrical signals are received from at least one, for example 2,3,4,5,6 or any larger number of electrodes, for example EEG electrodes, configured to record brain activity of two or more brain regions.

According to some exemplary embodiments, the received electrical signals, for example the received EEG electrical signals are processed at block 444. In some embodiments, the received electrical signals are filtered, for example to remove waves with undesired frequencies. Alternatively or additionally, the received EEG signals are filtered, for example to remove noise. In some embodiments, the received electrical signals are processed by a control circuitry, for example the control circuitry 314 shown in FIG. 3. Optionally, the received electrical signals are stored in a memory, for example memory 316 shown in FIG. 3, and the control circuitry processes at least some of the stored signals. In some embodiments, the electrical signals are received from electrodes positioned at locations 252, 254, 256 and 258 shown in FIG. 2B.

According to some exemplary embodiments, an anterior index, for example ASI is calculated at block 446. In some embodiments, the anterior index is calculated using electrical signals recorded only from anterior locations on the scalp, for example anterior location above anterior brain regions of different brain hemispheres. In some embodiments, the anterior score is calculated based on a relation between the recorded signals, for example a relation between signals recorded from anterior locations in different brain hemispheres, for example anterior locations 252 and 254 shown in FIG. 2B. In some embodiments, the anterior index is calculated by a control circuitry, for example control circuitry 314 shown in FIG. 3.

According to some exemplary embodiments, a global index, for example GSI, is calculated at block 448. In some embodiments, the global index is calculated using electrical signals recorded from anterior and posterior locations on the scalp. In some embodiments, the global index is calculated using electrical signals recorded from at least one anterior location and at least one posterior location on the scalp, each above a different brain hemisphere. In some embodiments, the global index is calculated based on a relation between the recorded signals, for example the electrical signals recorded from at least one anterior location and at least one posterior location on the scalp, each above a different brain hemisphere. In some embodiments, the global index is calculated based on a relation between electrical signals recorded from locations 252 and 258, or from locations 254 and 256 shown in FIG. 2B. In some embodiments, the anterior index is calculated by a control circuitry, for example control circuitry 314 shown in FIG. 3.

According to some exemplary embodiments, a relation between the calculated anterior index, for example ASI, and the calculated global index, for example GSI is optionally determined at block 450. In some embodiments, a score indicating the relation is calculated, for example by a control circuitry, for example control circuitry 314 shown in FIG. 3.

According to some exemplary embodiments, stroke or at least one parameter thereof is estimated at block 452, for example based on the calculated anterior index, or the determined relation between the calculated anterior index and the calculated global index.

According to some exemplary embodiments, penumbra or at least one parameter thereof is estimated at block 454, for example based on the calculated global index, or the determined relation between the calculated anterior index and the calculated global index.

According to some exemplary embodiments, an indication is generated and delivered at block 456. In some embodiments, the indication is generated and delivered by a user interface, for example user interface 318 shown in FIG. 3. In some embodiments, the indication is a human detectable indication, for example an audio and/or a visual indication. In some embodiments, the indication is transmitted by a communication circuitry, for example communication circuitry 320 to a remote device or to a neighboring device, for example a device located at a distance of up to 10 meters, for example up to 8 meters, up to 5 meters, up to 3 meters from the monitored subject. In some embodiments, the indication includes information regarding at least one of the calculated anterior index, the calculated global index, the estimated stroke or at least one parameter thereof and the estimated penumbra or at least one parameter thereof.

According to some exemplary embodiments, a treatment is optionally automatically modified or initiated at block 458. In some embodiments, the treatment is modified or initiated based on at least one of the calculated anterior index, the calculated global index, the estimated stroke or at least one parameter thereof and the estimated penumbra or at least one parameter thereof. In some embodiments, treatment is modified or initiated automatically, for example in case the stroke and/or penumbra estimating device includes a motorized injector for delivery of at least one bioactive compound to the subject.

### Exemplary Electrophysiological Measurements

According to some exemplary embodiments, the electrical signals, for example the EEG electrical signals are sampled for at least 5 minutes before and after each procedure, for example using saline based electrodes. In some embodiments, the saline embedded electrodes allow, for example, placing the electrodes on a head of a subject without the need for head shaving or gel use. Optionally, towels are used to support the head to obtain signal in the supine position. In some embodiments, data is sampled from channels F3, F4, O1, O2 and is optionally referenced to T3 for signal analysis, for example as shown in FIG. 2B showing an international 10-20 system.

### Exemplary Stimulus

According to some exemplary embodiments, a stimulation is delivered to the subject in a timed relation with the recording of the electrical signals, for example prior to and/or during the sampling of the signals. In some embodiments, the stimulation is delivered via a stimulation protocol configured to evoke brain activity, for example brain activity related to attention and perception. The oddball protocol is established as such, but other protocols might also be effective, for example other stimulation protocols used in anesthesia. In some embodiments, the stimulation comprises an auditory stimulation of pure tones, for example pure tones of 1000 Hz and of 2000 Hz at 60DB delivered, for example via earphones. In some embodiments, 80% of the stimulus has a 1000 Hz tone and 20% of the stimulus has a 2000 Hz tone. In some embodiments, the order of stimulus is arbitrary, for example presented every 2-3 seconds, with random delay in that range.

### Exemplary Signal Processing

Reference is now made to FIG. 5A depicting a signal processing procedure, according to some exemplary embodiments of the invention.

According to some exemplary embodiments, raw electrical signals, for example EEG signals, are sampled from at least two channels, at block 502. In some embodiments, the channels comprise channels F3, F4, from at least two anterior electrodes, and channels O1 and O2, from at least two posterior electrodes.

According to some exemplary embodiments, the raw electrical EEG signals are filtered to a delta band pass, at block 504. In some embodiments, a delta band pass has a frequency in a range of 1-4 Hz.

According to some exemplary embodiments, filtered samples including delta band pass from all the channels are divided into segments at block 506. In some embodiments, the segments are segments of 3-20 seconds, for example 5 seconds, 10 seconds or any intermediate, shorter or longer duration of segments. In some embodiments, each segment is further divided into epochs of 0.5-2 seconds, for example 0.7 seconds, 1 seconds, 1.2 seconds or any intermediate, shorter or longer time period for each epoch.

According to some exemplary embodiments, noise is evaluated in each sample, at block 508. In some embodiments, noise is evaluated in each sample at the 1 second epoch level, for example by computing the standard deviation/mean ratio.

According to some exemplary embodiments, noisy samples are discarded from further processing, at block 510. In some embodiments, epochs, which involved a ratio greater than 1, were considered noisy and were excluded from further analysis. Optionally, an epoch is excluded for all 4 electrodes, if it is considered noisy in even one of them. In some embodiments, 10-epoch segments, in which more than half (5) of the epochs were excluded as noisy are also excluded from further analysis.

According to some exemplary embodiments, if posterior activity (in electrodes O1 and O2) exceeded anterior activity (in electrodes F3 and F4) in the majority of the segment epochs, the segment is also excluded from further analysis. In some embodiments, poor EEG signal is defined for samples which do not include at-least 3 valid segments. In some embodiments, samples with poor EEG signal are disqualified from further analysis.

According to some exemplary embodiments, the samples are used for calculating an anterior index, for example ASI, at block 512, and/or a global index, for example GSI, at block 514.

### Exemplary Anterior Synchronization Index (ASI) Generation

Reference is now made to FIG. 5B depicting a process for generating an anterior index, for example ASI, according to some exemplary embodiments of the invention.

According to some exemplary embodiments, a correlation between delta activities recorded from anterior locations from different hemispheres, is calculated at block 520. In some embodiments, the correlation is calculated for each epoch. In some embodiments, the anterior locations, comprise locations 252 and 254 shown in FIG. 2B. In some embodiments, the correlation is calculated for the delta activities recorded from anterior locations only, and not for delta activities recorded from posterior locations.

According to some exemplary embodiments, a median is calculated for at least some epochs in each segment, at block 522, to generate a segment synchronization index. In some embodiments, the median is calculated at block 522 for all epochs in each segment, for example epochs of segments that include delta activity from anterior locations, for example F3 and F4 locations shown in FIG. 2B. In some embodiments, the medians calculated at block 522 for all valid, for example non-discarded and/or non-noisy, epochs.

According to some exemplary embodiments, a median is calculated for all segment synchronization indices, at block 524. In some embodiments, the median of all segment synchronization indices is calculated to generate an anterior index, for example an ASI.

A potential advantage of calculating a median may be to allow reduction of possible effect of aberrant noisy activity, when generating the anterior index, for example the ASI.

### Exemplary Global Synchronization Index (GSI) Generation

Reference is now made to FIG. 5C, depicting a process for generating a global synchronization index, for example GSI, according to some exemplary embodiments of the invention.

According to some exemplary embodiments, a crossed correlation between signals, is recorded from at least one anterior location and at least two posterior locations, each from a different brain hemisphere, is calculated for each epoch, at block 530. In some embodiments, the crossed correlation is calculated between signals having delta waves in a frequency of 1-4 Hz. In some embodiments, the crossed correlation is calculated between 2 or more combinations of electrodes located in anterior and posterior locations, for example between anterior left (F3) and posterior right (O2) and between anterior right (F4) and posterior left (O1), for example as shown in FIG. 2B.

According to some exemplary embodiments, a crossed correlation with the lowest value for each epoch is selected at block 532, for example to generate an epoch global synchronization index. In some embodiments, the crossed correlation with the lowest value is selected between the 2 or more combinations described at block 530, for example between a first combination of F3 and O2, and a second combination of F4 and O1.

According to some exemplary embodiments, a median is calculated for epoch global synchronization scores of a segment, at block 534, for example to generate a segment global synchronization index. In some embodiments, the median is calculated for a segment if at-least 2, 3, 4, 5 or any larger number of valid epochs, for example having a level of noise below a predetermined value, are identified for the segment.

According to some exemplary embodiments, a median of at least some or all valid segment global synchronization indices is calculated at block 536, for example to generate a global index, for example the GSI.

According to some exemplary embodiments, if, in all epochs of a segment, there was either only greater anterior activity, or alternatively if in all epochs of the segment, there was only greater posterior activity, the segment is considered as noisy, or non-valid.

### Exemplary Study I

### Background of the Exemplary Study I

Perioperative stroke incidence in high-risk surgical patients is 2-5% with associated mortality of up to 60%. Currently, there is no accepted brain monitoring for identification of stroke under anesthesia. Two evoked EEG based indices were developed; 1. Inter-hemispheric synchronization (IS) for correlation between the left and the right frontal hemispheres. and 2. Global synchronization (GS) index for correlation between whole brain regions. The IS and GS indices, are in the range of [0,1], where 1 indicates complete synchronization and 0 indicates complete desynchronization.

Due to the low incidence of stroke in the operating room, a special population was selected for the exemplary study I: the acute ischemic stroke (AIS) patients who undergo endo-vascular thrombectomy (EVT), and thus exhibit 3 clinical brain conditions during the procedure under anesthesia; Global penumbra, resolved stroke (NIHSS<4), definitive stroke (Stroke 2014). According to some exemplary embodiments, for example the embodiments shown in FIG. 1 and FIGS. 4A-4C, partial penumbra and/or partial stroke is estimated. In some embodiments, for example the embodiments shown in FIG. 1 and FIGS. 4A-4C, a degree of penumbra and/or a degree of stroke is estimated, for example based on a synchronization scale.

The study is small and only differentiates global penumbra from patents with at-least some degree of stroke. However, any relevant scale should be able to quantify the degree of penumbra vs. stroke.

The objectives of the Exemplary study I were: 1. using the IS index for identification of definitive stroke under anesthesia 2. using the GS index for differentiating between global penumbra and normal control under anesthesia.

### Methods of the Exemplary Study I

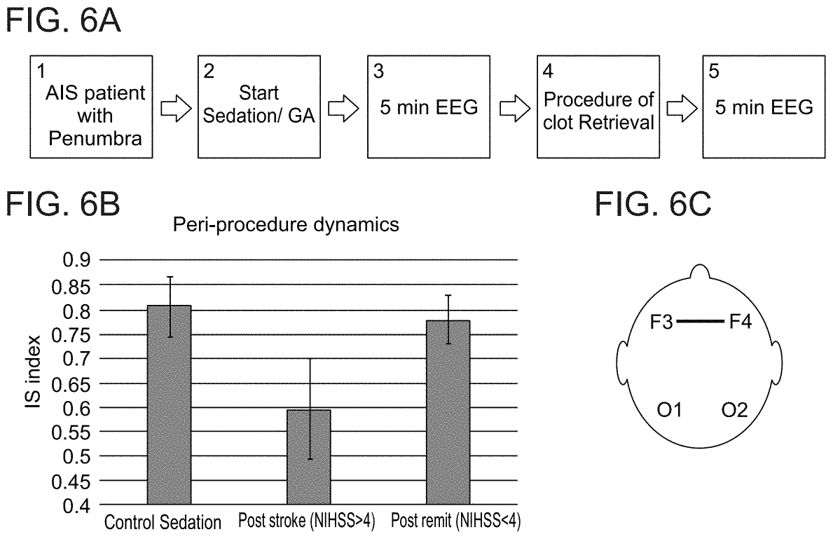

Twenty-four anesthetized patients undergoing EVT for AIS under sedation or general anesthesia (GA) were recruited. Evoked EEG was recorded, after induction of anesthesia, at the beginning and in the end of the thrombectomy, for example as shown in FIG. 6A. In the study and in some embodiments, for example embodiments described in FIGS. 4A-4C, the IS and GS indices were extrapolated based on algorithmic calculation of the EEG data. The control groups contained sedated patients with no known brain pathology.

### NIH Registration Number NCT02691338.

### Results of the Exemplary Study I

The IS index of normal sedated patients was (0.81±0.06, n=26) significantly higher than of patients with definitive stroke (NIHSS 13.6±7.6) under anesthesia (0.6±0.1, n=12, p<0.01). Furthermore, the IS index of patients who recovered (NIHSS≤4) at the end of the procedure (0.78±0.04, n=8), was significantly higher, compared to IS index of patients with definitive stroke (p<0.01), for example as shown in FIGS. 6B and 6C.

When NIHSS after procedure is up to 4 (post minor), the IS synchronization index increases to control levels under sedation/anesthesia. As shown in FIG. 6B, differences between post stroke and minor outcome/control groups is significant. In FIG. 6B, Anova: F(2,29)-24.59, P<0.0001. Tukey analysis: p<0.01 between post stroke and minor outcome/controls.

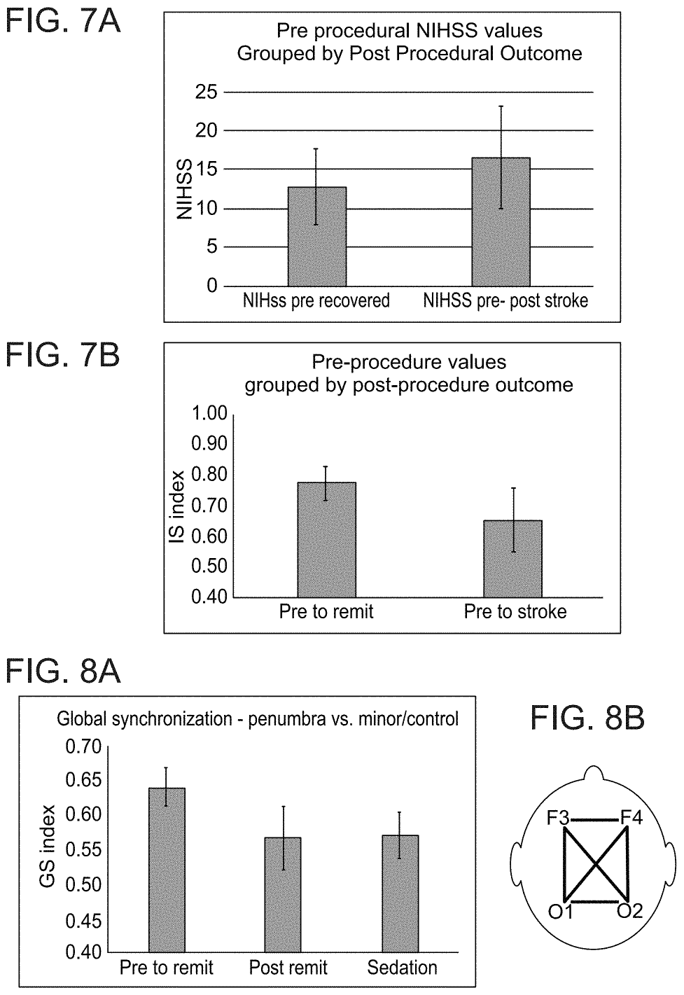

Moreover, the pre-treatment IS index of patients who recovered after the EVT (NIHSS<4), meaning they had a global penumbra, was significantly higher (0.77±0.05, n=6) when compared to IS index of patients who ended the procedure with definitive stroke (NIHSS 16.5±8) (0.65±0.1, n=12, p<0.01), for example as shown in FIGS. 7A and 7B.

FIGS. 7A and 7B describe that pre-procedure index differentiates between patients who end up with remission and patients with definitive stroke. In addition, the pre-procedure index of remitting patients is similar to minor outcome/no stroke patients. Complete penumbra might be in the same range as remission/no stroke. In FIGS. 7A and 7B, T-test, p<0.01. T-test between Pre NIHSS of recovered/Pre NIHSS to stroke is 0.08, no statistical significance.

However, the IS index of patients with global penumbra was not different from the IS index of normal sedated controls. Nevertheless, the GS index differentiated (p<0.01) between patients with Global penumbra (0.64±0.03) to normal sedated control (0.57±0.03) or to recovered patents (NIHSS<4) after the EVT (0.57±0.04), for example as shown in FIGS. 8A and 8B.

FIGS. 8A and 8B show that complete penumbra differs from remission/no stroke when using a global synchronization (GS) index. It is shown that the GS is reduced with remission/no stroke under sedation, but is increased in complete penumbra. This may be explained by no strict laterality asynchrony in complete penumbra, but there is also lack of focal differentiation. In FIGS. 8A and 8B, Anova: F(2,17)=6.2. Tukey: p<0.01/0.05 between pre to remit and post with post remit/control.

### Conclusions of the Exemplary Study I

This study indicates that: 1. IS index might identify patients with stroke under anesthesia, which manifests as decrease in interhemispheric synchronization. 2. GS index increase in the presence of global penumbra even if IS still did not decrease since presumably with penumbra there is reduced focal differentiation of activity, in comparison with the normal brain. The dual interpretation of IS and GS indices might differentiate between normal brain, global penumbra and stroke, for example as shown in table 1 below:

TABLE 1

| | | | |
|---|---|---|---|
| | | Global Penumbra | |
| | | (Potential for full | |
| | | recovery with | |
| | Control sedated | minimal | |
| | patients/Stroke | neurological | Definitive Stroke |
| Index | Remission (NIHSS<4) | damage, NIHSS<4) | (+/− Penumbra) |

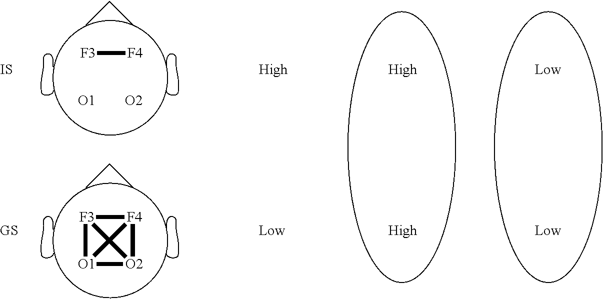

Table 1 shows IS and GS indices for identifying and differentiating between control/global penumbra and stroke patients.

In summary of the study and in some embodiments of the invention with respect to Global synchronization (GS):

### Minimal Synchrony Between Left Anterior and Right Posterior and Vice a Versa

In the study and in some embodiments of the invention, for example as shown in FIG. 1 and in FIGS. 4A-4C, GS is high (e.g. >0.6) in global penumbra and low in stroke, for example lower than 0.6. (even with partial penumbra) and in controls.
It is Very High (e.g. >0.7) with Epileptic Activity
2. Electrode placement 2 (beyond the P-O placement discussed before):

### With low NI2 (e.g. <0.75)

If it is a problem of brain injury (POCD/stroke/concussion/ . . . )— activity power will be consistently higher on one of the sides (e.g. >75% of the time)

### Exemplary Study II

### Abstract

Acute stroke under anesthesia is a dreadful complication, with a non-negligible prevalence. Importantly, over recent years there has been significant improvement in the ability to treat acute stroke. Rapid identification of acute stroke under anesthesia might lead to crucial and effective intervention. However, there seems to be conflicting evidence regarding the efficacy of the leading candidate technologies for this aim.

Notably, while the prevalence of acute stroke under anesthesia is non-negligible, it is nevertheless modest. It is necessary to sample a large number of patients, undergoing anesthesia, for the development and the establishment of technologies, which may identify acute stroke. This large sample requirement poses by itself a barrier upon the development of effective technologies. In a sense, the population of patients, who undergo thrombectomy under anesthesia or sedation, for acute stroke, may offer a “mirror image”. As for these patients, the pre-anesthetic status is of acute stroke and the post-anesthetic status is ranging from complete recovery to completed stroke. Thus, these patients should enable the monitoring of stroke-related dynamics with a much humbler sample.

In this pilot study (exemplary study II) it is shown, that an EEG delta synchrony index shows dynamics, which could be associated with the peri-thrombectomy stroke dynamics. Furthermore, there may also be synchronization findings, which may assist in the identification of penumbra.

### INTRODUCTION

Acute stroke under anesthesia is a dreadful complication, with a non-negligible prevalence, especially in susceptible clinical populations and certain types of operations. Importantly, over recent years there has been significant improvement in the ability to treat acute stroke with advances in thrombolytic pharmacological treatment and mechanical thrombectomy. Rapid identification of acute stroke under anesthesia might lead to crucial and effective intervention. In-fact, various technologies were evaluated for the identification of acute stroke under anesthesia (e.g. for NIRS). However, there seems to be conflicting evidence regarding the efficacy of the leading candidate technologies.

Notably, while the prevalence of acute stroke under anesthesia is non-negligible, it is nevertheless modest. It is important to sample a large number of patients, undergoing anesthesia, for the development and the establishment of technologies, which may identify acute stroke. This large sample requirement poses by itself a barrier upon the development of effective technologies for this aim.

Mechanical thrombectomy for acute stroke is being frequently performed under general anesthesia and sedation. In a sense, the population of patients, who undergo thrombectomy under anesthesia, for acute stroke, may offer a “mirror image” to the development of acute stroke under anesthesia. As for these patients, the pre-anesthetic status is of acute stroke and the post-anesthetic status is ranging from complete recovery to completed stroke. Thus, these patients should enable the monitoring of stroke-related dynamics with a much humbler sample, at-least at the initial phase of validating a technology for monitoring the dynamics of acute stroke under anesthesia. Certainly, once such a technology is developed, its relevancy for the operating room would require a large and conservative study of monitoring for stroke under anesthesia. However, as the purpose of this work was to provide an initial validation for the ability to identify stroke dynamics, this peri-thrombectomy dynamics was monitored under anesthesia and sedation.

Multiple focal electrophysiological indices were demonstrated to change after stroke. For example, there are long standing reports of focal differences in the power of specific frequency ranges and especially a decrease in the power of high frequency activity, with or without an increase in the low (delta−1-4 Hz) frequency activity (Nuwer et al., 1987; Schneider and Jordan, 2005). Such a focal differentiation could be identified by comparing the power of activity among various electrodes (Van Putten and Tavy, 2004). However, normal physiological changes would also lead to spatially differentiated changes in EEG power of activity (e.g. Benca et al., 1999). Furthermore, certain anesthesia effects may also lead to such spatially differentiated changes (e.g. alpha anteriorization—John et al., 2001 and spindles—Wolter et al., 2006). Thus, it is not trivial to offer a sensitive and specific marker for acute stroke under anesthesia on the basis of focal changes in power of activity.

Allegedly, comparison between the electrophysiological activity sampled by multiple electrodes might be desired to improve the likelihood for detecting smaller strokes. However, in practice the use of multi-electrode EEG setups is often too cumbersome for the busy clinical routine in anesthesia, and is even more problematic in the pressing timeline of acute stroke management. There would be an advantage for a simple system, which uses a minimal number of electrodes.

Over recent years it was suggested that the multi-channel electrophysiological signal is a superposition of two groups of processes, one which may be related to perception and the other, which may be related to attention (Shahaf and Pratt, 2013; Shahaf et al., 2015). Furthermore, it was shown that for the sake of monitoring the attention-related processes it suffices to analyze the delta activity (1-4 Hz) in a minimal number of electrodes (Shahaf et al., 2017; Shahaf et al., 2018a; Shahaf et al., 2018b; Isserles et al., 2018). Notably, it was also shown that such a marker could be monitored in real-time (Bartur et al., 2017; Bartur et al., 2019).

However, attention, and thereby attention-related markers, would be hindered by injury, which would damage multiple, if not all, neuropsychological processes, from sensation and perception to executive function. An index for attention might be affected by various brain injuries, regardless of the specific site of injury. Furthermore, we could also expect that a unilateral brain injury might impact the attention-related marker of the injured hemisphere to a greater extent, in comparison with the marker from the unimpacted (or less-impacted) hemisphere. Indeed, it was shown that by monitoring an attention-related marker and its asymmetry between the two frontal lobes, it is possible to identify the occurrence focal brain dysfunction (Shahaf et al., 2016). Notably, in the symmetry analysis inter-hemispheric synchronization was evaluated and not power differences, in order to overcome the normal physiological power differences, which were mentioned above.

Furthermore, in order to evoke sufficient activity of the attention-related processes and their electrophysiological manifestation, we often use a recruiting auditory oddball protocol (Shahaf et al., 2017; Shahaf et al., 2018a; Shahaf et al., 2018b). While using the auditory oddball, we analyze the signal in a task-related manner (Loo et al, 2009). In this method, the analysis is not time-locked to the stimulus, and is based on the assumption that the sustained attention level would be affected globally during the task.

The goal of this study was to evaluate whether our frontal delta synchrony index, for example ASI, would show dynamics, which could be associated with the peri-thrombectomy stroke dynamics. Specifically, it is hypothesized that clinical improvement would be associated with higher inter-hemispheric synchronization of the attention-related marker, and that a less favorable post-procedure outcome would be related with lower inter-hemispheric synchronization. Notably, interesting findings in this core analysis led us to some further post-hoc analyses as described below.

### Methods of Exemplary Study II

### Participants

The target group included 40 patients who were sampled before and after undergoing mechanical thrombectomy for acute ischemic stroke in the neuro-angio-suit. The control group included 15 patients undergoing sedation for non-neurological procedures in the angio-suit and in the cardiac-suit, with no known brain pathology. The data of this control group was taken from the first 15 samples of another study (NCT02938325). A sedation control group was selected since most of the catheterization procedures were done under sedation and not under general anesthesia. Of the 40 patients, which were sampled before and after mechanical thrombectomy, we obtained 16 valid (non-noisy, see below) pre-procedure samples and 15 valid post-procedure samples. Out of these samples, for 12 patients we obtained both pre- and post-procedure valid samples.

### Tools

### Neurological Evaluation

Each patient in the target group underwent full neurological examination upon arrival to the emergency room in the process of acute stroke diagnosis and within 24 hours after the procedure (and after the recovery from any possible effect of the procedural anesthesia or sedation). In most cases the neurological examination included a formal NIHSS evaluation. At other times, NIHSS could have been estimated on the basis of the report of the neurological examination.

### CTs and Angiography Scans

Each patient underwent CT, CT angio and CT perfusion scans as part of the process of acute stroke diagnosis and evaluation of applicability for catheterization. After the procedure, most patients underwent follow-up CT scans for evaluation. During the angiographic procedure the arterial flow before and after intervention was documented for each patient. See table 1 for details.

### Electrophysiological Measurement and the Auditory Oddball Protocol

EEG was sampled for at-least 5 minutes before and after each procedure by Emotiv Epoc 128 Hz system (using saline based electrodes) (Wang et al., 2015). The Emotiv Epoc since is a low-cost system, which could be placed quickly on the head even without prior experience in EEG sampling. The system uses saline embedded electrodes without the need for head shaving or gel use. Towels were used to support the head to obtain signal in the supine position. Data was sampled from channels F3, F4, O1, O2 and referenced to T7 for signal analysis.

According to some exemplary embodiments, for example the embodiments shown in FIG. 1 and in FIGS. 4A-4C, EEG is recorded from at least 1 EEG electrode, for example 2,4,6 or any larger number of electrodes while the subject is in an upright position or in a recumbent position. In some embodiments, the at least one electrode or EEG electrodes are part of a cap, a strap or a band positioned or attached to a head of the subject.

During the sampling period the patients were exposed to auditory stimulation of pure tones of 1000 Hz and of 2000 Hz at 60DB delivered via earphones. 80% of the stimulus were of the 1000 Hz tone and 20% of the stimulus were of the 2000 Hz tone. The order of stimulus was arbitrary, presented every 2-3 seconds, with random delay in that range (an auditory oddball protocol—which is often used in anesthesia in one format or the other—e.g. Kurita et al., 2001). Procedure

Each patient in the target group was sampled twice for at-least 5 minutes each time. The first sample was done after the onset of anesthesia or sedation and before the start of the procedure, and the second sample was done after the end of the procedure, while the patient was still fully anesthetized or sedated. For the control group, the EEG measurement started immediately after the onset of anesthesia and ended at the end of the procedure and before awakening the patient.

### Data Analysis

### Synchronization Indices—

### (I) Anterior Synchronization Index (ASI)

The raw EEG from two anterior electrodes (F3, F4) and two posterior electrodes (O1, O2) was filtered to the delta bandpass (1-4 Hz). The samples from the four channels were divided into 10 second segments and each segment was further divided into 1 second epochs. The sample from each of the electrodes was then evaluated for noisiness at the 1 second epoch level, by computing the standard deviation/mean ratio. Epochs, which involved a ratio greater than 1, were considered noisy (33,34) and were excluded from further analysis. An epoch was excluded for all 4 electrodes, if it was considered noisy in even one of them. 10-epoch segments, in which more than half (5) of the epochs were excluded as noisy were also excluded from further analysis. Furthermore, if posterior activity (in electrodes O1 and O2) exceeded anterior activity (in electrodes F3 and F4) in the majority of the segment epochs, the segment was also excluded from further analysis. Poor EEG signal was defined for samples which did not include at-least 3 valid segments. Samples with poor EEG signal were disqualified from further analysis. The average valid sample length (±s.d.) was 12(±5) minutes for the target group and 62(±28) minutes for the control group. The average valid segments count (±s.d.) was 14.10(±10.31) for the target group and 33.33(±25.66) for the control group.

We then computed the epoch anterior synchronization as the correlation between delta activities in F3 and F4. For each segment, a median anterior synchronization of all valid (non-noisy) epochs was computed. The anterior synchronization index was the median of all valid segment synchronization indices—using median and not mean was yet again another step in reducing a possible effect of aberrant noisy activity.

### (II) Global Synchronization Index (GSI)

Similar to the described above, the crossed delta synchronization between anterior left (F3) and posterior right (O2) and between anterior right (F4) and posterior left (O1) was computed for each epoch, and then selected the lowest between the two crossed synchronizations as the epoch global synchronization. Then we computed the segment global synchronization as the median of the valid epoch synchronizations comprising it, if at-least 5 valid epochs were identified for this segment. Finally, we computed the global synchronization index as the median of all valid segment global synchronizations. Importantly, if, in all epochs of the segment, there was either only greater anterior activity, or alternatively if in all epochs of the segment, there was only greater posterior activity, the segment was considered as noisy. Thus, for some segments there was an anterior synchronization value without a global synchronization value and vice versa. It should be stressed that all the analysis presented above, for both electrophysiological indices, was automatic without manual intervention.

### Sub-Grouping of the Target Group According to Clinical Outcome

For the sake of analyzing the association of the electrophysiological indices with clinical outcome, we divided the patients to two groups according to clinical outcome: (1) patients who after catheterization remitted or suffered from a minor stroke (NIHSS≤4) (Spilker et al., 1997) and (2) patients who after the procedure had a less favorable outcome (NIHSS≤4). The division to two groups was selected to enable statistical analysis, considering the modest sample size, and takes into account that for some patients the NIHSS evaluation was extracted post-hoc from the neurological examination record.

### Statistical Analysis

The major index we used for comparing among the study conditions—pre-catheterization, post-catheterization and sedation-control was the Anterior Synchronization Index (ASI). This comparison was based on one-way anova analysis with Tukey correction for multiple comparisons. The association between ASI and the clinical outcome (on its sub-groups, as described above), at the pre-catheterization and the post-catheterization measurements, was evaluated by a two-way Anova analysis.

### Results

### Association of Condition and Anterior Synchronization

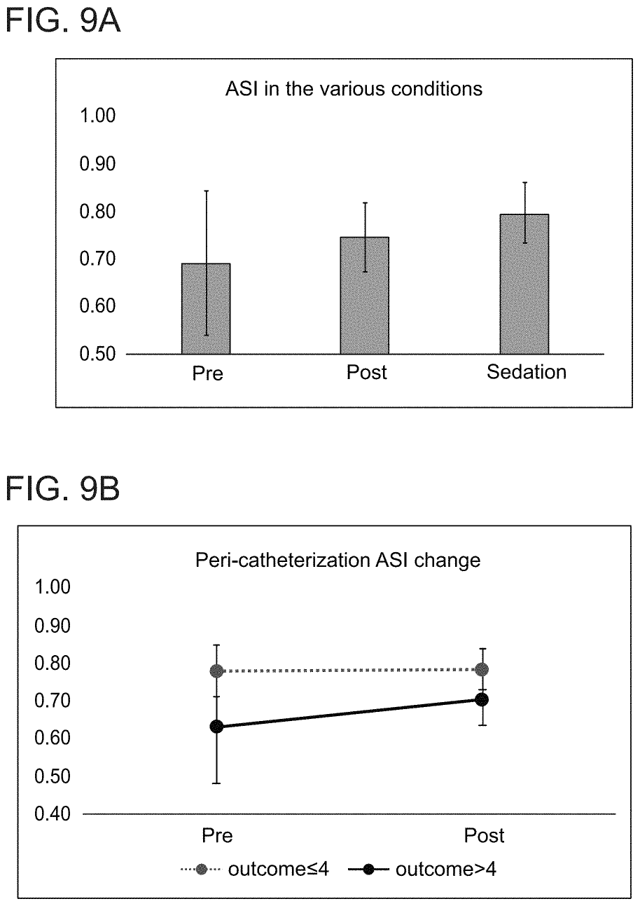

The anterior synchronization index (ASI) was valid, according to the above definitions, in 16 pre-catheterization samples, in 14 post-catheterization samples and in all 15 sedation-control samples. Over all the valid samples the ASI mean (±s.d.) was 0.69(±0.15) in the acute stroke samples prior to catheterization and 0.75(±0.07) after catheterization. In the sedation-control it was 0.80(±0.06), for example as shown in FIG. 9A. The difference was found significant by a one-way anova test [F(2,42),≈4.27, p<0.05]. After correction for multiple comparisons only the difference between the pre-catheterization samples and the sedation-control samples was found significant (p<0.05).

FIG. 9A shows ASI comparisons for major study conditions. Comparison of ASI among the pre-catheterization samples in acute stroke, the post-catheterization samples in acute stroke and the sedation-control samples

### Association of Outcome and Anterior Synchronization

We further analyzed the association between the neurological outcome after catheterization and the ASI. Due to the limited sample size we divided the patient's NIHSS outcome to two groups for the sake of comparison: the first group with post-procedural NIHSS up to 4 was defined as minor strokes and the second group with higher NIHSS was defined as moderate-severe strokes. We obtained 7 valid post-procedure samples with NIHSS≤4 and 7 valid post-procedure samples with NIHSS>4. The mean (±s.d.) ASI for the NIHSS≤4 post-procedure group was 0.79(±0.06) and for the NIHSS>4 post-procedure group it was 0.71(±0.07). Then, we also analyzed the pre-procedure samples based on this post-procedure outcome division between minor stroke and moderate-severe stroke. We obtained 6 valid pre-procedure samples with post-procedure NIHSS≤4 and 10 valid pre-procedure samples with post-procedure NIHSS>4. The mean (±s.d.) ASI for the NIHSS≤4 pre-procedure group was 0.78(±0.07) and for the NIHSS>4 pre-procedure group it was 0.63(±0.15), as shown in FIG. 9B summarizing all 4 values. For the sake of statistical analysis of the differences among these four conditions (pre-procedure samples who end with a minor stroke, pre-procedure samples who end with a moderate-severe stroke, post-procedure samples with minor stroke and post-procedure samples with moderate-severe stroke) we utilized a two-way anova test. We found that there was only significant difference with regard to the outcome [F(1,26),≈10.21, p<0.01]. We did not find significant differences when comparing pre- to post-procedure samples and did not find a significant interaction between the two variables. Thus, this result demonstrated association between ASI and outcome. However, interestingly, the difference according to post-procedure outcome was already observed in the samples prior to the procedure.

FIG. 9B shows ASI association with neurological outcome. ASI dynamics from before to after the catheterization of acute stroke patients. Samples are divided according to post-catheterization neurological outcome of minor stroke (NIHSS≤4) vs. moderate to severe stroke (NIHSS>4).

### Global Synchronization May Differentiate Between Penumbra and Normal Sedation

This finding led us to wonder post-hoc whether it is possible that penumbra-related conditions might also manifest with increased delta synchronization. As it is possible that patients, who ended up with minor stroke (or even remitted) had greater penumbra at the pre-procedure sample. In-fact, we wanted to evaluate in a very preliminary manner whether it might be possible to observe global “over-synchronization”, which could be related with penumbra. To this end we added the global synchronization index (GSI), which evaluates synchronization among all the 4 electrodes, which were used in this study—the two anterior ones and the two posterior ones. We then combined the GSI and ASI data for the various study conditions, as shown in table 2 below, and as could be seen in FIGS. 10A-10C, namely: for control-sedation (FIG. 10A) for post-procedure divided according to outcome (FIG. 10B) and to pre-procedure divided according to outcome (FIG. 10C). Note that only samples, for which we obtained both a valid ASI and a valid GSI, were included in these figures. While, as stated, this division of the sample seems too small for a meaningful statistical analysis, it might still be possible to observe some tendencies in the figures. Dividing the figures to quadrants, it might be possible to observe a higher ASI, but lower GSI in sedation-control (FIG. 10A) and also in post-procedure samples with minor stroke (FIG. 10B). Yet, for patients with a post-procedure moderate-severe stroke it seems that both indices are low (FIG. 10B) and this might already be evident for this group of patients at the pre-procedure samples (FIG. 10C). However, the pre-procedure samples, which end up as a minor stroke, and which might accord best with major penumbra, tend to have higher values for both indices, which might indicate global increased synchronization.

FIGS. 10A-10C show Spread of ASI and GSI values in the various study conditions, divided to quadrants. FIG. 10A shows Spread of the sedation-control patients with a tendency toward the higher ASI and lower GSI quadrant. FIG. 10B shows Spread of the post-catheterization samples. Minor stroke patients tend toward the higher ASI and lower GSI quadrant, similarly to sedation-control patients. Moderate-severe patients tend toward the lower ASI and GSI quadrant. FIG. 10C shows spread of the pre-catheterization samples. Patients with moderate-severe post-procedure outcome tend toward the lower ASI and GSI quadrant, similarly to their tendency in the post-catheterization samples. Patients with minor post-procedure stroke tend to the higher ASI and GSI quadrant in their pre-catheterization spread (penumbra?).

TABLE 2

| | | | | | | | | | | |
|---|---|---|---|---|---|---|---|---|---|---|
| Pre | | GS | GS | Post | | GS | GS | Sed | | |
| P# | AS | Outcome ≤ 4 | Outcome > 4 | P# | AS | Outcome ≤ 4 | Outcome > 4 | P# | AS | GS |

| | | | | | | | |
|---|---|---|---|---|---|---|---|
| 0.79 | 0.72 | | 0.85 | 0.70 | | 0.72 | 0.60 |
| 0.73 | | | 0.75 | | | 0.74 | 0.58 |
| 0.82 | 0.68 | | 0.76 | 0.55 | | 0.80 | 0.50 |
| 0.84 | 0.63 | | | 0.70 | | 0.71 | 0.65 |
| 0.67 | 0.54 | | 0.69 | 0.55 | | 0.71 | 0.51 |
| 0.84 | 0.68 | | 0.80 | 0.57 | | 0.80 | 0.71 |
| 0.75 | | 0.53 | 0.82 | 0.52 | | 0.84 | 0.72 |
| 0.60 | | 0.58 | 0.83 | | | 0.87 | 0.65 |
| 0.82 | | 0.77 | 0.67 | | | 0.87 | 0.57 |
| 0.67 | | 0.55 | 0.70 | | 0.66 | 0.76 | 0.58 |
| 0.71 | | | 0.62 | | 0.60 | 0.79 | 0.51 |
| 0.67 | | 0.66 | 0.71 | | 0.61 | 0.86 | 0.57 |
| 0.68 | | 0.62 | 0.84 | | 0.70 | 0.79 | 0.68 |
| 0.70 | | 0.62 | 0.72 | | | 0.78 | 0.56 |
| 0.40 | | | 0.68 | | 0.52 | 0.90 | 0.63 |
| 0.33 | | 0.53 | | | | | |

### Summary of Study

This work provided a feasibility demonstration to the sensitivity of an anterior delta synchronization index (ASI) to acute stroke under sedation and anesthesia. As we hypothesized, acute stroke seems to manifest in reduced synchronization. Furthermore, a basic association was also noted between neurological outcome (minor stroke vs. moderate and severe stroke) and the ASI. However, it is noted that patients with neurological dysfunction, but potentially also large penumbra, also demonstrate high ASI.

A possibility of over-synchronization in this condition was evaluated, using a global index of delta synchronization (GSI) among all sampled electrodes (F3, F4, O1 and O2). A potential tendency of GSI to be higher during the suspected large penumbra condition—of pre-catheterization sampling for patients who showed significant clinical improvement following the catheterization, reaching either complete remission or a minor stroke, is noted. Thus, ASI was similar between control patients under sedation, patients who remitted, or suffered only a minor stroke after catheterization and patients with plausibly large penumbra, but moderate or severe neurological deficit. But, GSI seemed to be higher in the penumbra condition, which could indicate over-synchronization in this condition.

Furthermore, if a sufficiently radiolucent EEG headset could be devised, it could also be used during catheterization for acute stroke to identify dynamics. This can teach us in real-time whether the relevant brain activity has normalized, or whether some further intervention should be considered. Effective markers for acute stroke and penumbra would also be interesting in non-anesthetized/sedated patients, such as in emergency triage at the emergency room or at the pre-hospital setting.

### References for Exemplary Study II

Bartur, G., Joubran, K., Peleg-Shani, S., Vatine, J. J., & Shahaf, G. (2017). An EEG tool for monitoring patient engagement during stroke rehabilitation: a feasibility study. BioMed research international, 2017.
Bartur G., Jourbran K., Peleg-Shani S., Vatine J. J., Shahaf G. A pilot study on the electrophysiological monitoring of patient's engagement in post-stroke physical rehabilitation. Disability and Rehabilitation: Assistive Technology. 2019. In print.
Benca, R. M., Obermeyer, W. H., Larson, C. L., Yun, B., Dolski, I., Kleist, K. D., . . . & Davidson, R. J. (1999). EEG alpha power and alpha power asymmetry in sleep and wakefulness. Psychophysiology, 36(4), 430-436.
Isserles, M., Daskalakis, Z. J., George, M. S., Blumberger, D. M., Sackeim, H. A., & Shahaf, G. (2018). Simple Electroencephalographic Treatment-Emergent Marker Can Predict Repetitive Transcranial Magnetic Stimulation Antidepressant Response—A Feasibility Study. The journal of ECT, 34(4), 274-282.
John, E. R., Prichep, L. S., Kox, W., Valdes-Sosa, P., Bosch-Bayard, J., Aubert, E., . . . & Gugino, L. D. (2001). Invariant reversible QEEG effects of anesthetics. Consciousness and cognition, 10(2), 165-183.
Kurita, T., Doi, M., Katoh, T., Sano, H., Sato, S., Mantzaridis, H., & Kenny, G. N. (2001). Auditory evoked potential index predicts the depth of sedation and movement in response to skin incision during sevoflurane anesthesia. Anesthesiology: The Journal of the American Society of Anesthesiologists, 95(2), 364-370.
Loo, S. K., Hale, T. S., Macion, J., Hanada, G., McGough, J. J., McCracken, J. T., & Smalley, S. L. (2009). Cortical activity patterns in ADHD during arousal, activation and sustained attention. Neuropsychologia, 47(10), 2114-2119.
Nuwer, M. R., Jordan, S. E., & Ahn, S. S. (1987). Evaluation of stroke using EEG frequency analysis and topographic mapping. Neurology, 37(7), 1153-1153.
Schneider, A. L., & Jordan, K. G. (2005). Regional attenuation without delta (RAWOD): a distinctive EEG pattern that can aid in the diagnosis and management of severe acute ischemic stroke. American journal of electroneurodiagnostic technology, 45(2), 102-117.
Shahaf, D. B., Shahaf, G., Mehta, J., & Venkatraghavan, L. (2016). Intracarotid etomidate decreases the interhemispheric synchronization in electroencephalogram (EEG) during the Wada test. Journal of neurosurgical anesthesiology, 28(4), 341-346.
Shahaf, G., Fisher, T., Aharon-Peretz, J., & Pratt, H. (2015). Comprehensive analysis suggests simple processes underlying EEG/ERP—demonstration with the go/no-go paradigm in ADHD. Journal of neuroscience methods, 239, 183-193.
Shahaf, G., Kuperman, P., Bloch, Y., Yariv, S., & Granovsky, Y. (2018). Monitoring Migraine Cycle Dynamics with an Easy-to-Use Electrophysiological Marker—A Pilot Study. Sensors, 18(11), 3918.
Shahaf, G., Nitzan, U., Erez, G., Mendelovic, S., & Bloch, Y. (2018). Monitoring attention in ADHD with an easy-to-use electrophysiological index. Frontiers in human neuroscience, 12, 32.
Shahaf, G., & Pratt, H. (2013). Thorough specification of the neurophysiologic processes underlying behavior and of their manifestation in EEG—demonstration with the go/no-go task. Frontiers in human neuroscience, 7, 305.
Shahaf, G., Yariv, S., Bloch, B., Nitzan, U., Segev, A., Reshef, A., & Bloch, Y. (2017). A pilot study of possible easy-to-use electrophysiological index for early detection of antidepressive treatment non-response. Frontiers in psychiatry, 8, 128.
Spilker, J., Kongable, G., Barch, C., Braimah, J., Bratina, P., Daley, S., . . . & Sailor, S. (1997). Using the NIH Stroke Scale to assess stroke patients. Journal of Neuroscience Nursing, 29(6), 384-393.
Van Putten, M. J., & Tavy, D. L. (2004). Continuous quantitative EEG monitoring in hemispheric stroke patients using the brain symmetry index. Stroke, 35(11), 2489-2492.
Wang, D., Mo, F., Zhang, Y., Yang, C., Liu, J., Chen, Z., & Zhao, J. (2015). Auditory evoked potentials in patients with major depressive disorder measured by Emotiv system. Bio-medical materials and engineering, 26(s1), S917-S923.
Wolter, S., Friedel, C., Bohler, K., Hartmann, U., Kox, W. J., & Hensel, M. (2006). Presence of 14 Hz spindle oscillations in the human EEG during deep anesthesia. Clinical neurophysiology, 117(1), 157-168.

It is expected that during the life of a patent maturing from this application many relevant EEG electrodes will be developed; the scope of the term EEG electrode is intended to include all such new technologies a priori.

As used herein with reference to quantity or value, the term “about” means “within ±10% off.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1-34. (canceled)

35. A method for evaluation of an ischemic region, comprising:
recording electrical signals from two or more brain regions of a subject;
determining a relation between activities of said two or more brain regions using said recorded electrical signals;
evaluating an ischemic region or at least one parameter of an ischemic region in said subject based on said determined relation;
repeating said recording, said determining and said evaluating for a time period of at least 30 minutes.

36. A method according to claim 35, comprising displaying an indication regarding said evaluated ischemic region or said at least one parameter of said ischemic region, in an operating room or in an emergency room.

37. A method according to claim 35, comprising:
filtering said recorded electrical signals to include signals with frequency values in a frequency range of 1-4 Hz, and wherein said determining comprises determining said relation based on said electrical signals with frequency values of 1-4 Hz.

38. A method according to claim 35, wherein said ischemic region comprises a penumbra, and wherein said evaluating comprises evaluating said penumbra or at least one parameter of said penumbra in said subject based on said determined relation.

39. A method according to claim 35, wherein said at least one ischemic region parameter comprises at least one of presence of an ischemic region, ischemic region size, stage and/or ischemic region location.

40. A method according to claim 35, wherein said at least one ischemic region parameter comprises stage of an ischemic region and/or characterization of ischemic region tissue.

41. A method according to claim 35, wherein said evaluating comprises evaluating a probability of tissue in the ischemic region to be rescued, if performing specific therapeutic procedures.

42. A method according to claim 41, wherein said specific therapeutic procedures comprise mechanical thrombectomy and/or pharmacological thrombolysis.

43. A method according to claim 35, wherein said evaluating comprises evaluating said ischemic region or said at least one parameter of said ischemic region in said subject based on said estimated determined relation and at least one additional measurement, wherein said at least one additional measurement comprises imaging, MRI, CT, NIRS, PET/SPECT, US-Doppler, MEG.

44. A method according to claim 35, wherein said recorded electrical signals are EEG electrical signals.

45. A method according to claim 35, comprising:
placing at least one electrode on a scalp of said subject, and moving said at least one electrode between different locations on the scalp for recording said electrical signals.

46. A method according to claim 35, comprising:
positioning two or more electrode on a scalp of a subject for recording said electrical signals.

47. A device for estimating an ischemic region or parameter thereof, comprising:
at least two EEG electrodes positioned on a scalp of a subject, wherein said at least two EEG electrodes are configured to record electrical signals from two different brain regions;
an EEG measurements unit connected to said at least two electrodes, and configured to receive EEG electrical signals from said at least two electrodes;
a memory;
a control circuitry connected to said EEG measurement unit and configured to estimate an ischemic region or a parameter thereof in said subject based on a relation between said received EEG electrical signals using at least one algorithm, lookup table or indications stored in said memory, wherein said EEG measurement unit receives said EEG electrical signals and wherein said control circuitry determines said relation and estimates said ischemic region or parameter thereof, repeatedly, during a time period of at least 30 minutes.

48. A device according to claim 47, wherein said control circuitry is configured to calculate a score indicating said determined relation, and wherein said ischemic region or parameter thereof is estimated based on said calculated score.

49. A device according to claim 47, wherein said at least two electrodes are part of a cap, a strap or a band configured to be attached to a head of said subject.

50. A device according to claim 47, wherein said control circuitry is configured to filter said received EEG electrical signals to include signals with frequency values of 1-4 Hz.

51. A method according to claim 35, comprising medicated, sedated or anesthetizing said subject prior to and/or during said evaluating, and wherein said recording comprises recording said electrical signals from two or more brain regions of said anesthetized subject, and wherein said evaluating comprises evaluating said ischemic region or at least one parameter of said ischemic region in said anesthetized subject based on said determined relation.

52. A method according to claim 51, comprising:
modifying care or stopping said anesthesia according to said evaluating results.

53. A method according to claim 35, wherein said two or more brain regions comprise at least one anterior brain region and at least one posterior brain region.

54. A method according to claim 35, wherein said two or more brain regions are brain regions in different hemispheres of the brain.
